(19) United States
(12) Patent Application Publication
(10) Pub. No.: US 2009/0111760 A1
(43) Pub. Date: Apr. 30, 2009

(54) MACROLONE COMPOUNDS

(76) Inventor: Catherine Simone Victoire Frydrych, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/718,529

(22) PCT Filed: Nov. 9, 2005
(86) PCT No.: PCT/EP2005/012037

§ 371 Date: May 3, 2007

(30) Foreign Application Priority Data
Nov. 11, 2004 (GB) 0424958.7

(51) Int. Cl. A61K 31/7048; C07H 17/08; C07H 17/00; A61K 31/7052; A61P 31/00

(52) U.S. Cl. 514/29; 536/7.3; 536/7.1

(57) ABSTRACT

A compound of formula (I)

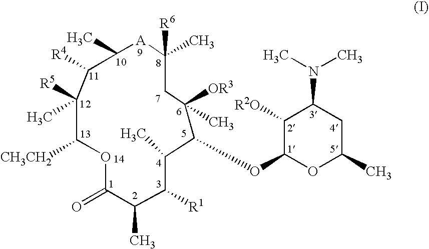

compositions comprising same, processes for their preparation and use of said compounds, particularly in the treatment of microbial infections.

The present invention relates to novel semi-synthetic macrolides having antimicrobial activity, in particular antibacterial activity. More particularly, the invention relates to 14- and 15-membered macrolides substituted at the 3 position, to processes for their preparation, to compositions containing them and to their use in medicine.

Macrolide antibacterial agents are known to be useful in the treatment or prevention of bacterial infections. However, the emergence of macrolide-resistant bacterial strains has resulted in the need to develop new macrolide compounds.

According to the present invention, we have now found novel 14- and 15-membered macrolides substituted at the 3 position which also have antimicrobial activity.

Thus, the present invention provides compounds of general formula (I)

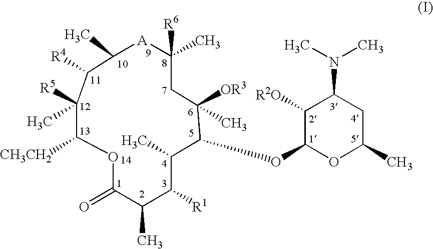

wherein

A is a bivalent radical —C(O)—, —N(R⁷)—CH₂—, —CH(NR⁸R⁹)— or —C(═NR¹⁰)—, or A and R⁴ taken together with the intervening atoms form a cyclic group having the following formula:

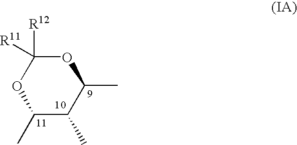

and R¹ is a group having the following formula:

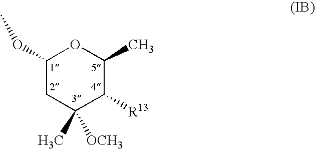

wherein R¹³ is —OC(O)(CH₂)_{d}U¹R¹⁴, —OC(O)N(R¹⁵)(CH₂)_{d}U¹R¹⁴, —O(CH₂)_{d}U¹R¹⁴,

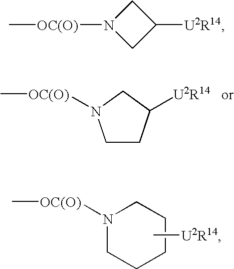

A is the bivalent radical —N(R⁷)—CH₂— and R¹ is a group having the following formula:

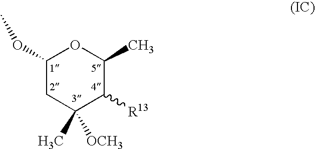

wherein R¹³ is —NHC(O)(CH₂)_{d}U¹R¹⁴;

R² is hydrogen or a hydroxyl protecting group;

R³ is hydrogen, C₁₋₄alkyl, or C₃₋₆alkenyl optionally substituted by 9- or 10-membered fused bicyclic heteroaryl;

R⁴ is hydroxy, C₃₋₆alkenyloxy optionally substituted by 9- or 10-membered fused bicyclic heteroaryl, or C₁₋₆alkoxy optionally substituted by C₁₋₆alkoxy or —O(CH₂)ₑNR⁷R¹⁶, or R⁴ and A taken together with the intervening atoms form a cyclic group of formula (IA),

R⁵ is hydroxy, or

R⁴ and R⁵ taken together with the intervening atoms form a cyclic group having the following formula:

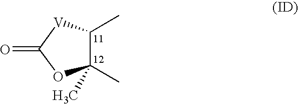

wherein V is a bivalent radical —CH₂—, —CH(CN)—, —O—, —N(R¹⁷)— or —CH(SR¹⁷)—, with the proviso that when R¹ is a group of formula (IC), V is —O—;

R⁶ is hydrogen or fluorine;

R⁷ is hydrogen or C₁₋₆alkyl;

R⁸ and R⁹ are each independently hydrogen, C₁₋₆alkyl or —C(O)R¹⁸, or

R⁸ and R⁹ together form ═CH(CR¹⁸R¹⁹)_{f}aryl, ═CH(CR¹⁸R¹⁹)_{f}heterocyclyl, ═CR¹⁸R¹⁹ or ═C(R¹⁸)C(O)OR¹⁸, wherein the alkyl, aryl and heterocyclyl groups are optionally substituted by up to three groups independently selected from R²⁰;

R¹⁰ is —OR²¹;

R¹¹ and R¹² are each independently hydrogen, C₁₋₆alkyl, heteroaryl, or aryl optionally substituted by one or two groups independently selected from hydroxyl and C₁₋₆alkoxy;

R¹⁴ is a heterocyclic group having the following formula:

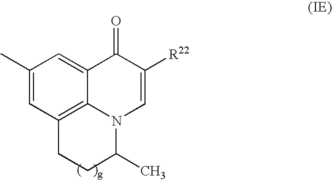

R¹⁵, R¹⁶, R¹⁸ and R¹⁹ are each independently hydrogen or C₁₋₆alkyl;

R¹⁷ is hydrogen or C₁₋₄alkyl optionally substituted by a group selected from optionally substituted phenyl, optionally substituted 5- or 6-membered heteroaryl and optionally substituted 9- or 10-membered fused bicyclic heteroaryl;

R²⁰ is halogen, cyano, nitro, trifluoromethyl, azido, —C(O)R²³, —C(O)OR²³, —OC(O)R²³, —OC(O)OR²³, —NR²⁴C(O)R²⁵, —C(O)NR²⁴R²⁵, —NR²⁴R²⁵, hydroxy, C₁₋₆alkyl, —S(O)ₕC₁₋₆alkyl, C₁₋₆alkoxy, —(CH₂)ᵢaryl or —(CH₂)ᵢheteroaryl, wherein the alkoxy group is optionally substituted by up to three groups independently selected from —NR¹⁸R¹⁹, halogen and —OR¹⁸, and the aryl and heteroaryl groups are optionally substituted by up to five groups independently selected from halogen, cyano, nitro, trifluoromethyl, azido, —C(O)R²⁶, —C(O)OR²⁶, —OC(O)OR²⁶, —NR²⁷C(O)R²⁸, —C(O)NR²⁷R²⁸, —NR²⁷R²⁸, hydroxy, C₁₋₆alkyl and C₁₋₆alkoxy;

R²¹ is hydrogen, C₁₋₆alkyl, C₃₋₇cycloalkyl, C₃₋₆alkenyl or a 5- or 6-membered heterocyclic group, wherein the alkyl, cycloalkyl, alkenyl and heterocyclic groups are optionally substituted by up to three groups independently selected from optionally substituted 5- or 6-membered heterocyclic group, optionally substituted 5- or 6-membered heteroaryl, —OR²⁹, —S(O)ⱼR²⁹, —NR²⁹R³⁰—CONR²⁹R³⁰, halogen and cyano;

R²² is —C(O)OR³¹, —C(O)NHR³¹, —C(O)CH₂NO₂ or —C(O)CH₂SO₂R⁷;

R²³ is hydrogen, C₁₋₁₀alkyl, —(CH₂)ₖaryl or —(CH₂)ₖheteroaryl;

R²⁴ and R²⁵ are each independently hydrogen, —OR¹⁸, C₁₋₆alkyl, —(CH₂)ₘaryl or —(CH₂)ₘheterocyclyl;

R²⁶ is hydrogen, C₁₋₁₀alkyl, —(CH₂)ₙaryl or —(CH₂)ₙheteroaryl;

R²⁷ and R²⁸ are each independently hydrogen, —OR¹⁸, C₁₋₆alkyl, —(CH₂)ₚaryl or —(CH₂)ₚheterocyclyl;

R²⁹ and R³⁰ are each independently hydrogen, C₁₋₄alkyl or C₁₋₄alkoxyC₁₋₄alkyl;

R³¹ is hydrogen,
C₁₋₆alkyl optionally substituted by up to three groups independently selected from halogen, cyano, C₁₋₄alkoxy optionally substituted by phenyl or C₁₋₄alkoxy, —C(O)C₁₋₆alkyl, —C(O)OC, alkyl, —OC(O)C, alkyl, —OC(O)OC₁₋₆alkyl, —C(O)NR³²R³³, —NR³²R³³ and phenyl optionally substituted by nitro or —C(O)OC₁₋₆alkyl,
—(CH₂)_{q}C₃₋₇cycloalkyl,
—(CH₂)_{q}heterocyclyl,
—(CH₂)_{q}heteroaryl,
—(CH₂)_{q}aryl,
C₃₋₆alkenyl, or
C₃₋₆alkynyl;

R³² and R³³ are each independently hydrogen or C₁₋₆alkyl optionally substituted by phenyl or —C(O)OC, alkyl, or

R³² and R³³, together with the nitrogen atom to which they are bound, form a 5- or 6-membered heterocyclic group optionally containing one additional heteroatom selected from oxygen, sulfur and N—R³⁴;

R³⁴ is hydrogen or methyl;

R³⁵ is hydrogen, C₁₋₄alkyl, C₃₋₇cycloalkyl, optionally substituted phenyl or benzyl, acetyl or benzoyl;

U¹ is a bivalent radical —W(CH₂)ᵣX—, —W(CH₂)ᵣ—, —W(CH₂)ᵣX(CH₂)ₛY—, —W(CH₂)ᵣX(CH₂)ₛ—, —W(CH₂)ᵣX(CH₂)ₛY(CH₂)ₜZ- or —W(CH₂)ᵣX(CH₂)ₛY(CH₂)ₜ—;

U² is U¹ or a bivalent radical —O—, —N(R³⁵)—, —S(O)ₙ— or —CH₂—;

W, X, Y and Z are each independently a bivalent radical —N(R³⁵)—, —O—, —S(O)ᵤ—, —N(R³⁵)C(O)—, —C(O)N(R³⁵)— or —N[C(O)R³⁵]—;

d is an integer from 2 to 5;

e is an integer from 2 to 4;

f, i, k, m, n, p and q are each independently integers from 0 to 4;

g is 0 or 1;

h, j and u are each independently integers from 0 to 2;

r, s and t are each independently integers from 2 to 5;

and pharmaceutically acceptable derivatives thereof.

The term “pharmaceutically acceptable” as used herein means a compound which is suitable for pharmaceutical use. Salts and solvates of compounds of the invention which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of the invention and their pharmaceutically acceptable salts and solvates.

The term “pharmaceutically acceptable derivative” as used herein means any pharmaceutically acceptable salt, solvate or prodrug, eg ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives. Examples of pharmaceutically acceptable derivatives are salts, solvates, esters, carbamates and phosphate esters. Additional examples of pharmaceutically acceptable derivatives are salts, solvates and esters. Further examples of pharmaceutically acceptable derivatives are salts and esters, such as salts.

The compounds of the present invention may be in the form of and/or may be administered as a pharmaceutically acceptable salt. For a review on suitable salts see Berge et al., J. Pharm. Sci., 1977, 66, 1-19.

Typically, a pharmaceutical acceptable salt may be readily prepared by using a desired acid or base as appropriate. The salt may precipitate from solution and be collected by filtration or may be recovered by evaporation of the solvent. For example, an aqueous solution of an acid such as lactobionic acid may be added to a solution of a compound of formula (I) in a solvent such as acetonitrile, acetone or THF, and the resulting mixture evaporated to dryness, redissolved in water and lyophilised to obtain the acid addition salt as a solid. Alternatively, a compound of formula (I) may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The skilled person will appreciate that where the compound of formula (I) contains more than one basic group bis salts (2:1 acid:compound of formula (I)) or tris salts (3:1 acid:compound of formula (I)) may also be formed and are salts according to the present invention.

Suitable addition salts are formed from inorganic or organic acids which form non-toxic salts and examples are lactobionate, mandelate (including (S)-(+)-mandelate, (R)-(−)-mandelate and (R,S)-mandelate), hydrochloride, hydrobromide, hydroiodide, sulfate, bisulfate, nitrate, phosphate, hydrogen phosphate, acetate, trifluoroacetate, maleate, malate, fumarate, lactate, tartrate, citrate, formate, gluconate, succinate, ethyl succinate (4-ethoxy-4-oxo-butanoate), pyruvate, oxalate, oxaloacetate, saccharate, benzoate, alkyl or aryl sulphonates (eg methanesulphonate, ethanesulphonate, benzenesulphonate or p-toluenesulphonate) and isethionate. In one embodiment, suitable salts include lactobionate, citrate, succinate, (L)-(+)-tartrate, (S)-(+)-mandalete and bis-(S)-(+)-mandalete, for example lactobionate, citrate, succinate and (L)-(+)-tartrate, such as lactobionate and citrate.

Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

Compounds of the invention may have both a basic and an acidic centre may therefore be in the form of zwitterions.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compounds of the invention are within the scope of the invention. The salts of the compound of formula (I) may form solvates (eg hydrates) and the invention also includes all such solvates.

The term “prodrug” as used herein means a compound which is converted within the body, eg by hydrolysis in the blood, into its active form that has medical effects. Pharmaceutically acceptable prodrugs are described in T. Higuchi and V. Stella, “Prodrugs as Novel Delivery Systems”, Vol. 14 of the A.C.S. Symposium Series; Edward B. Roche, ed., “Bioreversible Carriers in Drug Design”, American Pharmaceutical Association and Pergamon Press, 1987; and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of formula (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of formula (I). Further, in the case of a carboxylic acid (—COOH), esters may be employed, such as methyl esters, ethyl esters, and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

References hereinafter to a compound according to the invention include both compounds of formula (I) and their pharmaceutically acceptable derivatives.

With regard to stereoisomers, the compounds of formula (I) have more than one asymmetric carbon atom. In the general formula (I) as drawn, the solid wedge shaped bond indicates that the bond is above the plane of the paper. The broken bond indicates that the bond is below the plane of the paper. The wavy bond ( [symbol] ) indicates that the bond can be either above or below the plane of the paper. Thus, when R¹ is a group of formula (IC), the present invention includes both epimers at the 4″ carbon, and mixtures thereof.

It will be appreciated that the substituents on the macrolide may also have one or more asymmetric carbon atoms. Thus, the compounds of structure (I) may occur as individual enantiomers or diastereomers. All such isomeric forms are included within the present invention, including mixtures thereof.

Where a compound of the invention contains an alkenyl group, cis (Z) and trans (E) isomerism may also occur. The present invention includes the individual stereoisomers of the compound of the invention and, where appropriate, the individual tautomeric forms thereof, together with mixtures thereof.

Separation of diastereoisomers or cis and trans isomers may be achieved by conventional techniques, eg by fractional crystallisation, chromatography or H.P.L.C. A stereoisomeric mixture of the agent may also be prepared from a corresponding optically pure intermediate or by resolution, such as by H.P.L.C., of the corresponding mixture using a suitable chiral support or by fractional crystallisation of the diastereoisomeric salts formed by reaction of the corresponding mixture with a suitable optically active acid or base, as appropriate.

The compounds of formula (I) may be in crystalline or amorphous form. Furthermore, some of the crystalline forms of the compounds of structure (I) may exist as polymorphs, which are included in the present invention.

Compounds wherein R² represents a hydroxyl protecting group are in general intermediates for the preparation of other compounds of formula (I).

When the group OR² is a protected hydroxyl group this is conveniently an ether or an acyloxy group. Examples of particularly suitable ether groups include those in which R² is a trialkylsilyl (i.e. trimethylsilyl). When the group OR² represents an acyloxy group, then examples of suitable groups R² include acetyl or benzoyl.

When R¹³ is

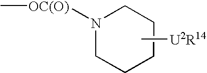

the —U²R¹⁴ group is typically attached at the 3- or 4-position on the piperidine ring.

The term “alkyl” as used herein as a group or a part of a group refers to a straight or branched hydrocarbon chain containing the specified number of carbon atoms. For example, C₁₋₁₀alkyl means a straight or branched alkyl containing at least 1, and at most 10, carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl. A C₁₋₄alkyl group is preferred, for example methyl, ethyl, n-propyl, isopropyl, n-butyl, isobutyl or t-butyl.

The term “C₃₋₇cycloalkyl” group as used herein refers to a non-aromatic monocyclic hydrocarbon ring of 3 to 7 carbon atoms such as, for example, cyclopropyl, cyclobutyl, cyclopentyl, cyclohexyl or cycloheptyl.

The term “alkoxy” as used herein refers to a straight or branched chain alkoxy group containing the specified number of carbon atoms. For example, C₁₋₆alkoxy means a straight or branched alkoxy containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy and hexyloxy. A C₁₋₄alkoxy group is preferred, for example methoxy, ethoxy, propoxy, prop-2-oxy, butoxy, but-2-oxy or 2-methylprop-2-oxy.

The term “alkenyl” as used herein as a group or a part of a group refers to a straight or branched hydrocarbon chain containing the specified number of carbon atoms and containing at least one double bond. For example, the term “C₂₋₆alkenyl” means a straight or branched alkenyl containing at least 2, and at most 6, carbon atoms and containing at least one double bond. Similarly, the term “C₃₋₆alkenyl” means a straight or branched alkenyl containing at least 3, and at most 6, carbon atoms and containing at least one double bond. Examples of “alkenyl” as used herein include, but are not limited to, ethenyl, 2-propenyl, 3-butenyl, 2-butenyl, 2-pentenyl, 3-pentenyl, 3-methyl-2-butenyl, 3-methylbut-2-enyl, 3-hexenyl and 1,1-dimethylbut-2-enyl. It will be appreciated that in groups of the form —O—C₂₋₆alkenyl, the double bond is preferably not adjacent to the oxygen.

The term “alkynyl” as used herein as a group or a part of a group refers to a straight or branched hydrocarbon chain containing the specified number of carbon atoms and containing at least one triple bond. For example, the term “C₃₋₆alkynyl” means a straight or branched alkynyl containing at least 3, and at most 6, carbon atoms and containing at least one triple bond. Examples of “alkynyl” as used herein include, but are not limited to, propynyl, 1-butynyl, 2-butynyl, 1-pentynyl and 3-methyl-1-butynyl.

The term “aryl” as used herein refers to an aromatic carbocyclic moiety such as phenyl, biphenyl or naphthyl, for example phenyl.

The term “heteroaryl” as used herein, unless otherwise defined, refers to an aromatic heterocycle of 5 to 10 members, having at least one heteroatom selected from nitrogen, oxygen and sulfur, and containing at least 1 carbon atom, including both mono and bicyclic ring systems. Examples of heteroaryl rings include, but are not limited to, furanyl, thiophenyl, pyrrolyl, pyrazolyl, imidazolyl, oxazolyl, isoxazolyl, thiazolyl, isothiazolyl, triazolyl, oxadiazolyl, tetrazolyl, thiadiazolyl, pyridyl, pyridazinyl, pyrazinyl, pyrimidinyl, triazinyl, quinolinyl, isoquinolinyl, 1,2,3,4-tetrahydroisoquinolinyl, benzofuranyl, benzimidazolyl, benzothienyl, benzoxazolyl, 1,3-benzodioxazolyl, indolyl, benzothiazolyl, furylpyridine, oxazolopyridyl and benzothiophenyl.

The term “5- or 6-membered heteroaryl” as used herein as a group or a part of a group refers to a monocyclic 5- or 6-membered aromatic heterocycle containing at least one heteroatom independently selected from oxygen, nitrogen and sulfur. Examples include, but are not limited to, furanyl, thiophenyl, pyrrolyl, pyrazolyl, imidazolyl, oxazolyl, isoxazolyl, thiazolyl, isothiazolyl, triazolyl, oxadiazolyl, tetrazolyl, pyridyl, pyridazinyl, pyrazinyl, pyrimidinyl and triazinyl.

The term “9- or 10-membered fused bicyclic heteroaryl” as used herein as a group or a part of a group refers to a 9- or 10-membered fused bicyclic heteroaryl containing at least one heteroatom selected from oxygen, nitrogen and sulphur. Examples include, but are not limited to, quinolinyl, isoquinolinyl, 1,2,3,4-tetrahydroisoquinolinyl, benzofuranyl, benzimidazolyl, benzothienyl, benzoxazolyl, 1,3-benzodioxazolyl, indolyl, benzothiazolyl, furylpyridine, oxazolopyridyl and benzothiophenyl.

The term “heterocyclyl” as used herein, unless otherwise defined, refers to a monocyclic or bicyclic 3- to 10-membered saturated or non-aromatic, unsaturated hydrocarbon ring containing at least one heteroatom selected from oxygen, nitrogen and sulfur. Preferably, the heterocyclyl ring has five or six ring atoms. Examples of heterocyclyl groups include, but are not limited to, pyrrolidinyl, tetrahydrofuranyl, tetrahydrothiophenyl, imidazolidinyl, pyrazolidinyl, piperidyl, piperazinyl, morpholino, tetrahydropyranyl and thiomorpholino.

The term “5- or 6-membered heterocyclic group” as used herein as a group or part of a group refers to a monocyclic 5- or 6-membered saturated hydrocarbon ring containing at least one heteroatom independently selected from oxygen, nitrogen and sulfur. Examples of such heterocyclyl groups include, but are not limited to, pyrrolidinyl, tetrahydrofuranyl, tetrahydrothiophenyl, imidazolidinyl, pyrazolidinyl, piperidyl, piperazinyl, morpholino, tetrahydropyranyl and thiomorpholino.

The term “halogen” refers to a fluorine, chlorine, bromine or iodine atom.

The terms “optionally substituted phenyl”, “optionally substituted phenyl or benzyl”, “optionally substituted 5- or 6-membered heteroaryl”, “optionally substituted 9- or 10-membered fused bicyclic heteroaryl” or “optionally substituted 5- or 6-membered heterocyclic group” as used herein refer to a group which is substituted by 1 to 3 groups independently selected from halogen, C₁₋₄alkyl, C₁₋₄alkoxy, hydroxy, nitro, cyano, amino, C₁₋₄alkylamino or diC₁₋₄alkylamino, phenyl and 5- or 6-membered heteroaryl.

As the skilled person will appreciate, the compounds of formula (I) are derivatives of known 14- and 15-membered macrolides derived from erythromycin A that have antibacterial activity and contain a cladinose moiety with a hydroxy group or amino group at 4″ position. The 14- and 15-membered macrolides which may be derivatised according to the invention include, for example, the following:
azithromycin,
11-O-methyl-azithromycin,
azithromycin 11,12-carbonate,
6-O-methyl erythromycin A,
6-O-methyl erythromycin A 9-oxime,
6-O-methyl erythromycin A 9-O-alkyl-oximes,
erythromycin 9-oxime,
erythromycin 9-O-alkyl-oximes,
erythromycin 11,12-carbonate,
erythromycin 9-oxime 11,12-carbonate,
6-O-methyl-11-deoxy-11-amino-erythromycin A 11,12-carbamate,
(9S)-9-dihydro-erythromycin, and
(9S)-9-dihydro-erythromycin 9,11-ethylidene acetal,
   and the above macrolides in which the 4″ hydroxyl group is replaced by an amino group to give a macrolide having (R), (S) or (R,S) stereochemistry at the 4″ position.

In the compounds of formula (I), the heterocyclic group of formula (IE) (R¹⁴) is attached to the 4″ position of the 14- or 15-membered macrolide via a linker chain. Linker chains suitable for use according to the present invention include, for example, the following:

—OC(O)(CH₂)₂NH(CH₂)₂S—; and

—OC(O)(CH₂)₂NH(CH₂)₂S(O)₂—.

Representative examples of A include —C(O)—, —N(R⁷)—CH₂— and —C(═NR¹⁰)—.

A representative example of R¹ is

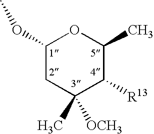

A representative example of R² is hydrogen.

Representative examples of R³ include hydrogen and C₁₋₄alkyl, such as hydrogen and methyl, for example hydrogen.

In one embodiment, R⁴ and R⁵ are hydroxy, or R⁴ and R⁵ taken together with the intervening atoms form a cyclic group having the following structure:

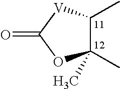

wherein V is a bivalent radical selected from —CH₂—, —CH(CN)—, —O—, —N(R¹⁷)— or —CH(SR¹⁷)—.

In a further embodiment, R⁴ and R⁵ are hydroxy.

A representative example of R⁶ is hydrogen.

A representative example of R⁷ is C₁₋₄alkyl, for example methyl.

In one embodiment, R¹¹ and R¹² are each independently hydrogen or C₁₋₆alkyl. In a further embodiment, one of R¹¹ and R¹² is hydrogen and the other is methyl.

A representative example of R¹³ is —OC(O)(CH₂)_{d}U¹R¹⁴.

A representative example of R¹⁴ is a heterocyclic group having the following formula:

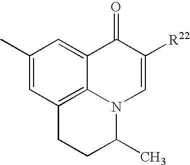

In one embodiment, R¹⁵ is hydrogen.

In one embodiment, R¹⁷ is hydrogen.

Representative examples of R²¹ include hydrogen and C₁₋₄alkyl optionally substituted by —OR²⁹, for example hydrogen and methyl optionally substituted by —OR²⁹.

In one embodiment, R²² is —C(O)OR³¹, —C(O)NHR³¹ or —C(O)CH₂NO₂. A representative example of R²² is —C(O)OR³¹.

A representative example of R²⁹ is C₁₋₄alkyl, for example methyl.

A representative example of R³¹ is hydrogen.

A representative example of R³⁵ is hydrogen.

In one embodiment, U¹ is —W(CH₂)ᵣX— or —W(CH₂)ᵣ—. A representative example of U¹ is —W(CH₂)ᵣX—.

A representative example of V is —O—.

A representative example of W is —N(R³⁵)—.

A representative example of X is —S(O)ₛ—.

A representative examples of d is 2.

A representative example of g is 1.

A representative example of r is 2.

Representative examples of u include 0 and 2.

It is to be understood that the present invention covers all combinations of the embodiments and representative examples described hereinabove. It is also to be understood that the present invention encompasses compounds of formula (I) in which a particular group or parameter, for example R⁷, R¹⁸, R¹⁹, R²⁰, R²³, R²⁴, R²⁵, R²⁶, R²⁷, R²⁸, R²⁹, R³⁰, R³², R³³, R³⁴, R³⁵, h, i, j, k, m, n, p and u may occur more than once. In such compounds it will be appreciated that each group or parameter is independently selected from the values listed.

Compounds of the invention include:
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-6-O-methyl-lerythromycin A,
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-azithromycin,
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-azithromycin-11,12-carbonate,
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-erythromycin A (9E)-oxime,
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-erythromycin A (9E)-methoxymethyloxime,
4″-O-{[2-({2-[(2-carboxy-5-(R,S)-methyl-1-oxo-6,7-dihydro-1H,5H-pyrido[3,2,1-ij]quinolin-9-yl)sulfonyl]ethyl}amino)ethyl]propionyl}-6-O-methyl-erythromycin A,
4″-O-{[2-({2-[(2-carboxy-5-(R,S)-methyl-1-oxo-6,7-dihydro-1H,5H-pyrido[3,2,1-ij]quinolin-9-yl)sulfonyl]ethyl}amino)ethyl]propionyl}-azithromycin,
4″-O-{[2-({2-[(2-carboxy-5-(R,S)-methyl-1-oxo-6,77-dihydro-1H,5H-pyrido[3,2,1-ij]quinolin-9-yl)sulfonyl]ethyl}amino)ethyl]propionyl}-erythromycin A (9E)-methoxime,
4″-O-{[2-({2-[(2-carboxy-5-(R,S)-methyl-1-oxo-6,7-dihydro-1H,5H-pyrido[3,2,1-ij]quinolin-9-yl)sulfonyl]ethyl}amino)ethyl]propionyl}-erythromycin A (9E)-methoxymethyloxime,
4″-O-{[2-({2-[(2-carboxy-5-(*)-methyl-1-oxo-6,7-dihydro-1H,5H-pyrido[3,2,1-ij]quinolin-9-yl)thio]ethyl}amino)ethyl]propionyl}-6-O-methyl-erythromycin A,
4″-O-{[2-({2-[(2-carboxy-5-(*)-methyl-1-oxo-6,7-dihydro-1H,5H-pyrido[3,2,1-ij]quinolin-9-yl)thio]ethyl}amino)ethyl]propionyl}-6-O-methyl-erythromycin A,
4″-O-{[2-(2-{2-[(2-carboxy-5-(R,S)-methyl-1-oxo-6,7-dihydro-1H,5H-pyrido[3,2,1-ij]quinolin-9-yl)amino]ethyl}oxy(ethylamino))]propionyl}-6-O-methyl-erythromycin A,
4″-O-{[2-({2-[(2-carboxy-5-(R,S)-methyl-1-oxo-6,7-dihydro-1H,5H-pyrido[3,2,1-ij]quinolin-9-yl)amino]ethyl}amino)]propionyl}-6-O-methyl-erythromycin A,
4″-O-{[2-(2-{2-[(2-carboxy-5-(R,S)-methyl-1-oxo-6,7-dihydro-1H,5H-pyrido[3,2,1-ij]quinolin-9-yl)amino]ethyl}oxy(ethylamino))]carbamoyl}-6-O-methyl-erythromycin A,
4″-O-{[2-({2-[(2-carboxy-5-(R,S)-methyl-1-oxo-6,7-dihydro-1H,5H-pyrido[3,2,1-ij]quinolin-9-yl)amino]ethyl}amino)]propionyl}-6-O-methyl-erythromycin A (9E)-methoxymethyloxime,
4″-O-{[2-({2-[(2-carboxy-5-(R,S)-methyl-1-oxo-6,7-dihydro-1H,5H-pyrido[3,2,1-ij]quinolin-9-yl)amino]ethyl}amino)]propionyl}-6-O-methyl-erythromycin A (9E)-oxime,
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)]ethyl}-6-O-methyl-erythromycin A,
4″-O-{[2-({2-[(2-carboxy-5-(R,S)-methyl-1-oxo-6,7-dihydro-1H,5H-pyrido[3,2,1-ij]quinolin-9-yl)thio]ethyl)methylamino)]ethyl}-6-O-methyl-erythromycin A,
4″-O-{[2-({2-[(2-carboxy-5-(R,S)-methyl-1-oxo-6,7-dihydro-1H,5H-pyrido[3,2,1-ij]quinolin-9-yl)thio]ethyl}methylamino)]ethyl}-azithromycin,
4″-O-{[2-({2-[(2-Carboxy-5-(R,S)-methyl-1-oxo-6,7-dihydro-1H,5H-pyrido[3,2,1-ij]quinolin-9-yl)thio]ethyl}ethylamino)]ethyl}-azithromycin,
4″-O-{2-[2-(2-Carboxy-5-(R,S)-methyl-6,7-dihydro-1H,5H-pyrido[3,2,1-ij]-1-oxo-quinolin-9-ylthio)ethoxy]ethylcarbamoyl}-erythromycin A 9(E)-O-methoxymethyloxime,
4″-O-{2-[3-(2-Carboxy-5-(R,S)-methyl-1-oxo-6,7-dihydro-1H,5H-pyrido[3,2,1-ij]-quinolin-9-yl)propyloxy]ethylcarbamoyl}-6-O-methyl-erythromycin A,
4″-O-{2-[3-(2-Carboxy-5-(R,S)-methyl-1-oxo-6,7-dihydro-1H,5H-pyrido[3,2,1-ij]-quinolin-9-yl)propyloxy]ethylcarbamoyl}-erythromycin A 9(E)-O-methoxymethyl oxime,
4″-O-{2-[2-(2-Carboxy-5-(R,S)-methyl-6,7-dihydro-1H,5H-pyrido[3,2,1-ij]-1-oxo-quinolin-9-ylthio)ethoxy]ethylcarbamoyl}-6-O-methyl-erythromycin A,
4″-O-{2-[2-(2-Carboxy-5-(R,S)-methyl-6,7-dihydro-1H,5H-pyrido[3,2,1-ij]-1-oxo-quinolin-9-ylthio)ethoxy]ethylcarbamoyl}-erythromycin A 9(E)-oxime,
4″-O-{2-[3-(2-Carboxy-5-(R,S)-methyl-1-oxo-6,7-dihydro-1H,5H-pyrido[3,2,1-ij]-quinolin-9-yl)propyloxy]ethylcarbamoyl}-azithromycin,
4″-O-{2-[2-(2-Carboxy-5-(R,S)-methyl-6,7-dihydro-1H,5H-pyrido[3,2,1-ij]-1-oxo-quinolin-9-ylthio)ethoxy]ethylcarbamoyl}-azithromycin,
4″-O-{2-[2-(2-Carboxy-1-oxo-6,7-dihydro-1H,5H-pyrido[3,2,1-ij]-quinolin-9-yl)propyloxy]ethylcarbamoyl}-erythromycin A 9(E)-O-cyanomethyl oxime,
4″-O-{2-[3-(5-Carboxy-6-oxo-1,2-dihydro-6H-pyrrolo[3,2,1-ij]quinolin-8-yl)propyloxy]ethylcarbamoyl}-6-O-methyl-erythromycin A,
4″-O-{2-[3-(5-Carboxy-6-oxo-1,2-dihydro-6H-pyrrolo[3,2,1-ij]quinolin-8-yl)propyloxy]ethylcarbamoyl}-erythromycin A 9(E)-O-methoxy methyloxime,
4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-methoxy methyloxime,
4″-O-{2-[2-(5-Carboxy-6-oxo-1,2-dihydro-6H-pyrrolo[3,2,1-ij]quinolin-8-ylthio)ethoxy]ethylcarbamoyl}-6-O-methyl-erythromycin A,
4″-O-{2-[3-(5-Carboxy-6-oxo-1,2-dihydro-6H-pyrrolo-[3,2,1-ij]-quinolin-8-yl)propyloxy]ethylcarbamoyl}-azithromycin,
4″-O-{2-[2-(5-Carboxy-6-oxo-1,2-dihydro-6H-pyrrolo-[3,2,1-ij]-quinolin-8-ylthio)ethoxy]ethylcarbamoyl}-azithromycin,
4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-oxime, and
4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-cyano methyloxime,
   and pharmaceutically acceptable derivatives thereof.

Compounds of the invention also include:
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-azithromycin-11,12-carbonate,
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-erythromycin A (9E)-oxime,
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-erythromycin A (9E)-methoxymethyloxime and
4″-O-{[2-({2-[(2-carboxy-5-(*)-methyl-1-oxo-6,7-dihydro-1H,5H-pyrido[3,2,1-ij]quinolin-9-l)thio]ethyl}amino)ethyl]propionyl}-6-O-methyl-erythromycin A,
   and pharmaceutically acceptable derivatives thereof.

One or more compounds according to the invention exhibit antimicrobial activity, in particular antibacterial activity, against a range of clinical pathogenic microorganisms. Using a standard microtiter broth serial dilution test, one or more of the compounds of the invention have been found to exhibit useful levels of activity against a range of pathogenic microorganisms. For example, the compounds of the invention may be active against strains of Staphylococcus aureus, Streptococcus pneumoniae, Moraxella catarrhalis, Streptococcus pyogenes, Haemophilus influenzae, Enterococcus faecalis, Chlamydia pneumoniae, Mycoplasma pneumoniae and Legionella pneumophila. The compounds of the invention may also be active against resistant strains, for example erythromycin resistant strains. Thus, for example, the compounds of the invention may be active against erythromycin resistant strains of Streptococcus pneumoniae, Streptococcus pyogenes and Staphylococcus aureus.

The compounds of the invention may therefore be used for treating a variety of diseases caused by pathogenic microorganisms, in particular bacteria, in human beings and animals. It will be appreciated that reference to treatment includes acute treatment or prophylaxis as well as the alleviation of established symptoms.

Compounds of the invention may have different levels of antibacterial activity against different strains of the same bacteria.

Thus, according to another aspect of the present invention we provide a compound of formula (I) or a pharmaceutically acceptable derivative thereof for use in therapy.

According to a further aspect of the invention we provide a compound of formula (I) or a pharmaceutically acceptable derivative thereof for use in the treatment or prophylaxis of systemic or topical microbial infections in a human or animal body.

According to a further aspect of the invention we provide the use of a compound of formula (I) or a pharmaceutically acceptable derivative thereof in the manufacture of a medicament for use in the treatment or prophylaxis of systemic or topical microbial infections in a human or animal body.

According to a yet further aspect of the invention we provide a method of treatment of the human or non-human animal body to combat microbial infections comprising administration to a body in need of such treatment of an effective amount of a compound of formula (I) or a pharmaceutically acceptable derivative thereof.

Infections include, but are not limited to, infections of soft tissue such as skin, for example acne or impetigo.

While it is possible that, for use in therapy, a compound of the invention may be administered as the raw chemical it is preferable to present the active ingredient as a pharmaceutical formulation eg when the agent is in admixture with a suitable pharmaceutical excipient, diluent or carrier selected with regard to the intended route of administration and standard pharmaceutical practice.

Accordingly, in one aspect, the present invention provides a pharmaceutical composition or formulation comprising a compound of the invention or a pharmaceutically acceptable derivative thereof in association with a pharmaceutically acceptable excipient, diluent and/or carrier. The excipient, diluent and/or carrier must be “acceptable” in the sense of being compatible with the other ingredients of the formulation and not deleterious to the recipient thereof.

In another aspect, the invention provides a pharmaceutical composition comprising, as active ingredient, a compound of the invention or a pharmaceutically acceptable derivative thereof in association with a pharmaceutically acceptable excipient, diluent and/or carrier for use in therapy, and in particular, in the treatment of human or animal subjects suffering from a condition susceptible to amelioration by an antimicrobial compound.

In another aspect, the invention provides a pharmaceutical composition comprising a therapeutically effective amount of the compounds of the present invention and a pharmaceutically acceptable excipient, diluent and/or carrier (including combinations thereof).

There is further provided by the present invention a process of preparing a pharmaceutical composition, which process comprises mixing a compound of the invention or a pharmaceutically acceptable derivative thereof, together with a pharmaceutically acceptable excipient, diluent and/or carrier.

The compounds of the invention may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable excipients, diluents and/or carriers. Acceptable excipients, diluents and carriers for therapeutic use are well known in the pharmaceutical art, and are described, for example, in Remington's Pharmaceutical Sciences, Mack Publishing Co. (A. R. Gennaro edit. 1985). The choice of pharmaceutical excipient, diluent and/or carrier can be selected with regard to the intended route of administration and standard pharmaceutical practice. The pharmaceutical compositions may comprise as—or in addition to—the excipient, diluent and/or carrier any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, eg as a carrier, diluent or solubiliser. Alpha-, beta- and gamma-cyclodextrins are most commonly used and suitable examples are described in WO 91/11172, WO 94/02518 and WO 98/55148.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see international patent application No. WO 02/00196 (SmithKline Beecham).

The routes for administration (delivery) include, but are not limited to, one or more of: oral (eg as a tablet, capsule, or as an ingestable solution), topical, mucosal (eg as a nasal spray or aerosol for inhalation), nasal, parenteral (eg by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual.

There may be different composition/formulation requirements depending on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by: inhalation, in the form of a suppository or pessary; topically in the form of a lotion, solution, cream, ointment or dusting powder; transdermally, for example, by a skin patch; orally in the form of tablets containing excipients such as starch or lactose, as capsules or ovules either alone or in admixture with excipients; in the form of elixirs, solutions or suspensions containing flavouring or colouring agents; or as a parenterally formulation, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Thus compositions of the invention include those in a form especially formulated for parenteral, oral, buccal, rectal, topical, implant, ophthalmic, nasal or genito-urinary use. For some applications, the agents of the present invention are delivered systemically (such as orally, buccally, sublingually), more preferably orally. Hence, preferably the agent is in a form that is suitable for oral delivery.

If the compound of the present invention is administered parenterally, then examples of such administration include one or more of: intravenously, intraarterially, intraperitoneally, intrathecally, intraventricularly, intraurethrally, intrasternally, intracranially, intramuscularly or subcutaneously administering the agent, and/or by using infusion techniques.

For parenteral administration, the compound is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The compounds according to the invention may be formulated for use in human or veterinary medicine by injection (eg by intravenous bolus injection or infusion or via intramuscular, subcutaneous or intrathecal routes) and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilising, solubilising and/or dispersing agents. Alternatively the active ingredient may be in sterile powder form for reconstitution with a suitable vehicle, eg sterile, pyrogen-free water, before use.

The compounds of the invention can be administered (eg orally or topically) in the form of tablets, capsules, ovules, elixirs, solutions or suspensions, which may contain flavouring or colouring agents, for immediate-, delayed-, modified-, sustained-, pulsed- or controlled-release applications.

The compounds of the invention may also be presented for human or veterinary use in a form suitable for oral or buccal administration, for example in the form of solutions, gels, syrups, mouth washes or suspensions, or a dry powder for constitution with water or other suitable vehicle before use, optionally with flavouring and colouring agents. Solid compositions such as tablets, capsules, lozenges, pastilles, pills, boluses, powder, pastes, granules, bullets or premix preparations may also be used. Solid and liquid compositions for oral use may be prepared according to methods well known in the art. Such compositions may also contain one or more pharmaceutically acceptable carriers and excipients which may be in solid or liquid form.

Some compounds of the invention may be more suitable for use in a particular type of formulation/administration route than others.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The compounds of the invention may also be administered orally in veterinary medicine in the form of a liquid drench such as a solution, suspension or dispersion of the active ingredient together with a pharmaceutically acceptable carrier or excipient.

The compounds of the invention may also, for example, be formulated as suppositories eg containing conventional suppository bases for use in human or veterinary medicine or as pessaries eg containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (eg eye ear or nose drops) or pour-ons.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water.

Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds may also be dermally or transdermally administered, for example, by use of a skin patch.

For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

As indicated, the compound of the present invention can be administered intranasally or by inhalation and is conveniently delivered in the form of a dry powder inhaler or an aerosol spray presentation from a pressurised container, pump, spray or nebuliser with the use of a suitable propellant, eg dichlorodifluoromethane, trichlorofluoromethane, dichlorotetrafluoroethane, a hydrofluoroalkane such as 1,1,1,2-tetrafluoroethane (HFA 134AT″″) or 1,1,1,2,3,3,3-heptafluoropropane (HFA 227EA), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, eg using a mixture of ethanol and the propellant as the solvent, which may additionally contain a lubricant, eg sorbitan trioleate.

Capsules and cartridges (made, for example, from gelatin) for use in an inhaler or insufflator may be formulated to contain a powder mix of the compound and a suitable powder base such as lactose or starch.

For topical administration to the lungs by inhalation, the compounds according to the invention may be delivered for use in human or veterinary medicine via a nebuliser.

The compounds of the invention may also be used in combination with other therapeutic agents. The invention thus provides, in a further aspect, a combination comprising a compound of the invention or a pharmaceutically acceptable derivative thereof together with a further therapeutic agent.

It will be appreciated that the amount of a compound of the invention required for use in treatment will vary with the nature of the condition being treated and the age and the condition of the patient and will be ultimately at the discretion of the attendant physician or veterinarian.

When a compound of the invention or a pharmaceutically acceptable derivative thereof is used in combination with a second therapeutic agent active against the same or a different disease state (for example, topical administration with other active ingredients such as corticosteroids or antifungals as appropriate) the dose of each compound may differ from that required when the compound is used alone. Appropriate doses will be readily appreciated by those skilled in the art.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, for example by methods known for such compounds in the art.

The compositions may contain from 0.01-99% of the active material. For topical administration, for example, the composition will generally contain from 0.01-10%, more preferably 0.01-1% of the active material.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the characteristics of the individual undergoing therapy.

For oral and parenteral administration to humans, the daily dosage level of the agent may be in single or divided doses.

For systemic administration the daily dose as employed for adult human treatment it will range from 2-100 mg/kg body weight, preferably 5-60 mg/kg body weight, which may be administered in 1 to 4 daily doses, for example, depending on the route of administration and the condition of the patient. When the composition comprises dosage units, each unit will preferably contain 200 mg to 1g of active ingredient. The duration of treatment will be dictated by the rate of response rather than by arbitrary numbers of days, for example 3, 7, 14 or 21 days.

Compounds of general formula (I) and pharmaceutically acceptable derivatives thereof may be prepared by the general methods outlined hereinafter, said methods constituting a further aspect of the invention. In the following description, the groups R¹ to R³⁵, U¹, U², V, W, X, Y, Z, d, e, f, g, h, i, j, k, m, n, p, q, r, s, t and u have the meaning defined for the compounds of formula (I) unless otherwise stated.

The groups U^{1z}R^{14z}, U^{2z}R^{14z}, X^{z}R^{14z} and R^{14z} are U¹R¹⁴, U²R¹⁴, XR¹⁴ and R¹⁴ as defined for formula (I) or groups convertible to U¹R¹⁴, U²R⁴, XR¹⁴ and R¹⁴. Conversion of a group U^{1z}R^{14z}, U^{2z}R^{14z}, X^{z}R^{14z} or R^{14z} to a U¹R¹⁴, U²R¹⁴, XR¹⁴ or R¹⁴ group typically arises if a protecting group is needed during the reactions described below. A comprehensive discussion of the ways in which such groups may be protected and methods for cleaving the resulting protected derivatives is given by for example T. W. Greene and P. G. M Wuts in Protective Groups in Organic Synthesis 2^{nd} ed., John Wiley & Son, Inc 1991 and by P. J. Kocienski in Protecting Groups, Georg Thieme Verlag 1994 which are incorporated herein by reference. Examples of suitable amino protecting groups include acyl type protecting groups (eg formyl, trifluoroacetyl and acetyl), aromatic urethane type protecting groups (eg benzyloxycarbonyl (Cbz) and substituted Cbz, and 9-fluorenylmethoxycarbonyl (Fmoc)), aliphatic urethane protecting groups (eg tert-butyloxycarbonyl (Boc), isopropyloxycarbonyl and cyclohexyloxycarbonyl) and alkyl type protecting groups (eg benzyl, trityl and chlorotrityl). Examples of suitable oxygen protecting groups may include for example alkyl silyl groups, such as trimethylsilyl or tert-butyldimethylsilyl; alkyl ethers such as tetrahydropyranyl or tert-butyl; or esters such as acetate. Hydroxy groups may be protected by reaction of for example acetic anhydride, benzoic anhydride or a trialkylsilyl chloride in an aprotic solvent. Examples of aprotic solvents are dichloromethane, N,N-dimethylformamide, dimethylsulfoxide, tetrahydrofuran and the like.

The compounds of general formula (I) and derivatives thereof may be purified by conventional methods known in the art. For example, the compounds may be purified by HPLC using an aqueous solution of an acid such as formic acid with an organic co-solvent such as acetonitrile or methanol.

In one embodiment of the invention, compounds of formula (I) wherein R¹³ is —OC(O)(CH₂)_{d}U¹R¹⁴ and d is an integer from 2 to 5 may be prepared by reaction of a 4″ hydroxy compound of formula (II), wherein R² may be a hydroxy protecting group, with a suitable activated and protected derivative of the carboxylic acid (III), followed where necessary by subsequent removal of the hydroxyl protecting group R² and conversion of the U^{1z}R^{14z} group to U¹R¹⁴.

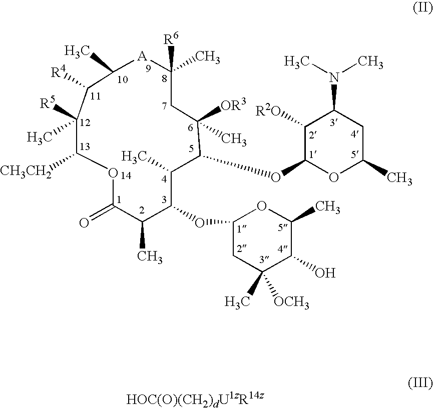

Compounds of formula (I) wherein R¹³ is —NHC(O)(CH₂)_{d}U¹R¹⁴ and d is an integer from 2 to 5 may be prepared by reaction of a 4″ amine compound of formula (IIA) with a carboxylic acid compound of formula (IIIA), or a suitable activated and protected derivative thereof, followed where necessary by subsequent conversion of the U^{1z}R^{14z} group to U¹R¹⁴.

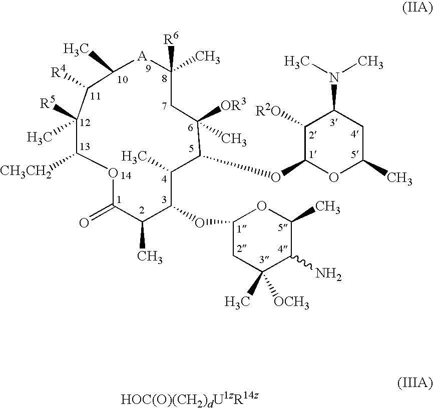

Suitable activated derivatives of the carboxyl group in the compounds of formula (III) or (IIIA) include the corresponding acyl halide, mixed anhydride or activated ester such as a thioester. The reaction is preferably carried out in a suitable aprotic solvent such as a halohydrocarbon (eg dichloromethane) or N,N-dimethylformamide optionally in the presence of a tertiary organic base such as dimethylaminopyridine or triethylamine or in the presence of inorganic base (eg sodium hydroxide) and at a temperature within the range of 0° to 120° C. The compounds of formula (II) or (IIA) and (III) or (IIIA) may also be reacted in the presence of a carbodiimide such as dicyclohexylcarbodiimide (DCC).

In another embodiment of the invention, compounds of formula (I) wherein R¹³ is —OC(O)N(R⁵)(CH₂)_{d}U¹R¹⁴,

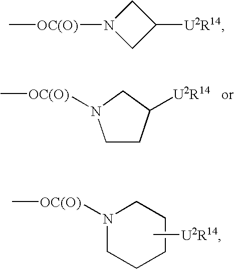

may be prepared by reaction of a suitable activated compound of formula (IIB) wherein R² is optionally a hydroxy protecting group and R³⁸ is an activating group such as imidazolyl or halogen, with a suitable protected derivative of an amine (IV), (IVA), (IVB) or (IVC), followed where necessary by subsequent removal of the hydroxyl protecting group R² and conversion of the U^{1z}R^{14z} or U^{2z}R^{14z} group to U¹R¹⁴ or U²R¹⁴.

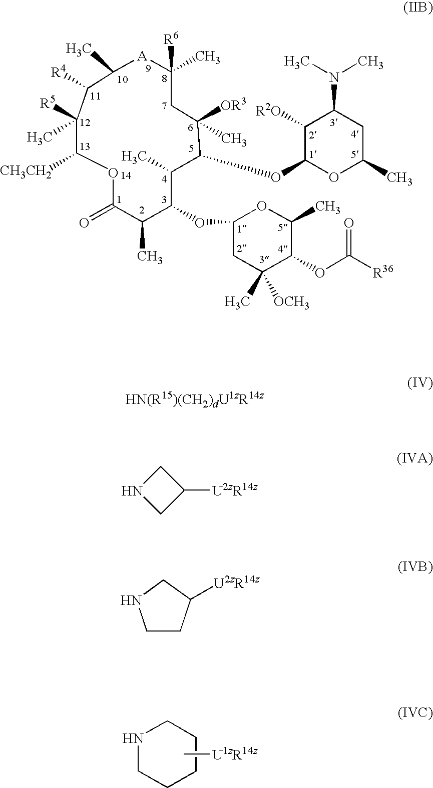

The reaction is preferably carried out in a suitable aprotic solvent such as N,N-dimethylformamide in the presence of an organic base such as 1,8-diazabicyclo[5.4.0]undec-7-ene (DBU).

In another embodiment of the invention, compounds of formula (I) wherein R¹³ is —O(CH₂)_{d}U¹R¹⁴, U¹ is —W(CH₂)ᵣX— or —W(CH₂)ᵣ—, and W is —N(R³⁵)— may be prepared by reaction of a 4″ aldehyde compound of formula (IIC) wherein A, R⁴ and R⁵ may be suitably protected and d′ is an integer from 1 to 4, with a suitable protected derivative of the amine (V) or (VA), followed where necessary by subsequent removal of the hydroxyl protecting group R² and conversion of the X^{z}R^{14z} or R^{14z} group to XR¹⁴ or R¹⁴.

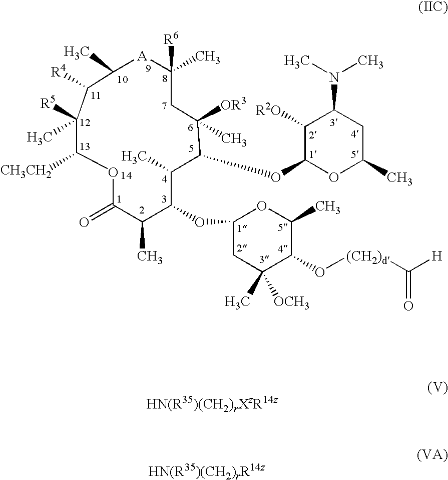

The reductive amination reaction is preferably carried out in a solvent such as methanol and DMF under neutral to mildly acidic conditions. A suitable reducing agent is, for example, sodium cyanoborohydride, and suitable reagents for adjusting acidity are acetic acid and sodium acetate.

Compounds of formula (IIC) where d′ is 1 may be prepared from suitably protected compounds of formula (VI) by oxidative cleavage for example using osmium tetroxide and sodium periodate. Where d′ is 2, hydroboration of suitably protected compounds of formula (VI) with 9-BBN, or other suitable boranes, followed by treatment with peroxide and then oxidation yields compounds of formula (IIC), d′ is 2. For d′=3 or 4, compounds of formula (VI) may be chain extended using olefin cross-metathesis (H. E. Blackwell et. al. J. Am. Chem. Soc., 2000, 122, 58-71) with a suitably functionalised olefin, for example but-2-ene-1,4-diol, followed by double bond reduction and oxidation of the terminal alcohol. Compounds of formula (VI) can be formed by palladium-catalysed allylation of suitably protected 4″ hydroxy compounds, for example when A is —C(O)—, by 2′,11-bis-silylation and conversion of the 9-ketone to a bicyclic ketal by interaction with the 12-OH and an alcohol, for example methanol.

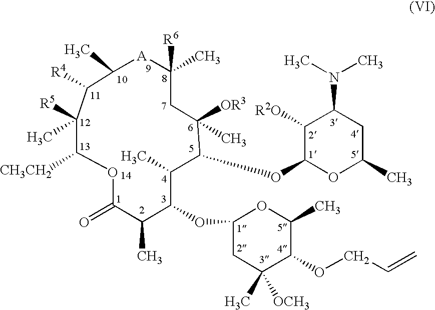

In another embodiment of the invention, compounds of formula (I) wherein R¹³ is —OC(O)(CH₂)_{d}U¹R¹⁴, d is an integer from 2 to 5, U¹ is —W(CH₂)ᵣX—, and W is —N(R³⁵)—, —O— or —S—, may be prepared by reaction of compounds of formula (VII)

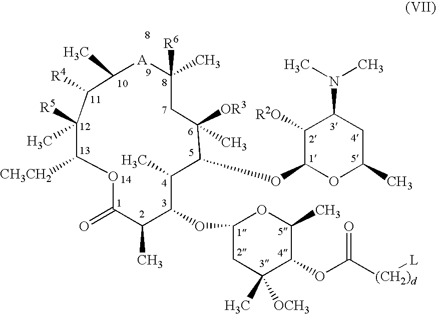

wherein d is an integer from 2 to 5 and L is a suitable leaving group, with HU^{1z}R^{14z} (VIII) in which W is —N(R³⁵)—, —O— or —S—.

Similarly, compounds of formula (I) wherein R¹³ is —OC(O)N(R¹⁵)(CH₂)_{d}U¹R¹⁴, U¹ is —W(CH₂)ᵣX— or —W(CH₂)ᵣ—, and W is —N(R³⁵)— or —S—, may be prepared by reaction of compounds of formula (VIIA)

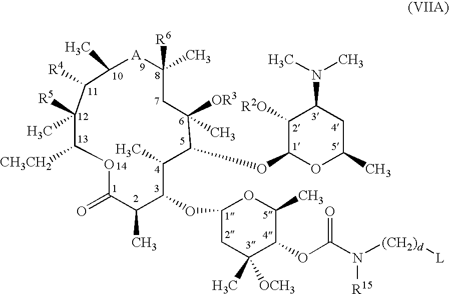

wherein d is an integer from 2 to 5 and L is a suitable leaving group, with HU^{1z}R^{14z} (VIII) in which W is —N(R³⁵)— or —S—.

Similarly, compounds of formula (I) wherein R¹³ is —O(CH₂)_{d}U¹R¹⁴, U¹ is W(CH₂)ᵣX or —W(CH₂)ᵣ—, and W is —N(R³⁵)— or —S—, may be prepared by reaction of compounds of formula (VIIB)

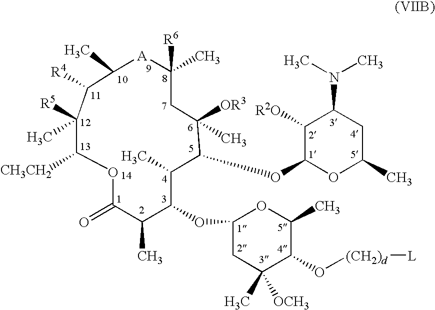

wherein d is an integer from 2 to 5 and L is a suitable leaving group, with HU^{1z}R^{14z} (VIII) in which W is —N(R³⁵)— or —S—.

Further, compounds of formula (I) wherein R¹³ is —NHC(O)(CH₂)_{d}U¹R¹⁴, d is an integer from 2 to 5, U¹ is —W(CH₂)ᵣX— or —W(CH₂)ᵣ—, and W is —N(R³⁵)— or —S—, may be prepared by reaction of compounds of formula (VIIC)

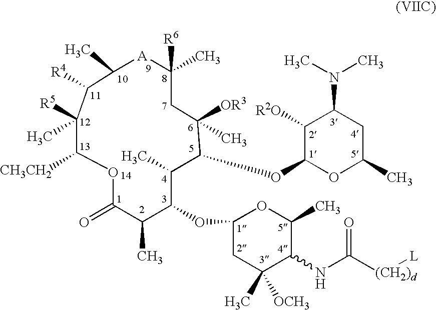

wherein d is an integer from 2 to 5 and L is a suitable leaving group, with HU^{1z}R^{14z} (VIII) in which W is —N(R³⁵)— or —S—.

The reaction between (VII), (VIIA), (VIIB) or (VIIC) and (VIII) is preferably carried out in a solvent such as a halohydrocarbon (eg dichloromethane), an ether (eg tetrahydrofuran or dimethoxyethane), acetonitrile or ethyl acetate and the like, dimethylsulfoxide, N,N-dimethylformamide or 1-methyl-pyrrolidinone and in the presence of a base, followed, if desired, by removal of the hydroxyl protecting group R² and conversion of the U^{1z}R^{14z} group to U¹R¹⁴. Examples of the bases which may be used include organic bases such as diisopropylethylamine, triethylamine and 1,8-diazabicyclo[5.4.0]undec-7-ene, and inorganic bases such as potassium hydroxide, cesium hydroxide, tetraalkylammonium hydroxide, sodium hydride and potassium hydride. Suitable leaving groups for this reaction include halide (eg chloride, bromide or iodide) or a sulfonyloxy group (eg tosyloxy or methanesulfonyloxy).

Compounds of formula (VII) and (VIIC) may be prepared by reaction of a compound of formula (II) or (IIA), wherein R² is a hydroxyl protecting group, with a suitable activated derivative of the carboxylic acid HOC(O)(CH₂)_{d}L (IX), wherein L is a suitable leaving group as above defined. Suitable activated derivatives of the carboxyl group are those defined above for carboxylic acids (III) or (IIIA). The reaction is carried out using the conditions described above for the reaction of a compound of formula (II) or (IIA) with carboxylic acid (III) or (IIIA).

In another embodiment of the invention, compounds of formula (I) wherein R¹³ is —O(CH₂)_{d}U¹R¹⁴, U¹ is —O(CH₂)ᵣX— or —O(CH₂)ᵣ—, may be prepared by reaction of compounds of formula (X)

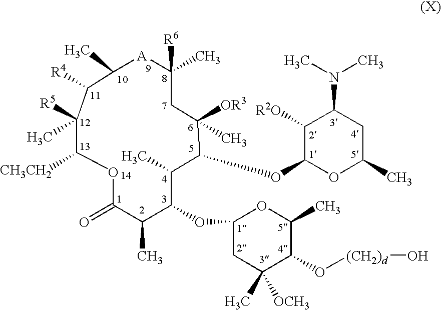

wherein d is an integer from 2 to 5 with a suitable compound of formula HU^{1z}R^{14z} (VIII), for example a compound of formula (XI)

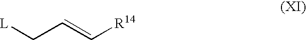

in which L is a suitable leaving group, in the presence of a catalyst such as tetrakis(triphenylphosphine) palladium.

In a preferred embodiment of the invention, compounds of formula (I) wherein R¹³ is —OC(O)(CH₂)_{d}U¹R¹⁴, d is 2, U¹ is as above defined, and W is —N(R³⁵)— or —S—, may be prepared by Michael reaction of a compound of formula (XII), wherein R² is optionally a hydroxyl protecting group

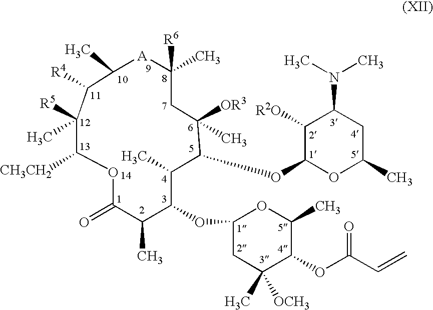

with a compound of formula HU^{1z}R^{14z} (VIII). The reaction is suitably carried out in a solvent such as dimethylsulfoxide, N,N-dimethylformamide, 1-methyl-pyrrolidinone, a halohydrocarbon (eg dichloromethane), an ether (eg tetrahydrofuran or dimethoxyethane), acetonitrile or alcohol (e.g methanol or isopropanol) and the like, and in the presence of a base, followed, if desired, by removal of hydroxyl protecting group R² and conversion of the U^{1z}R^{14z} group to U¹R¹⁴. Similarly, compounds of formula (I) wherein R¹³ is —OC(O)(CH₂)_{d}U¹R¹⁴, d is 2, U¹ is as above defined and Y is —O— may also be prepared by Michael reaction in a solvent such as dimethylsulfoxide, N,N-dimethylformamide, 1-methyl-pyrrolidinone, a halohydrocarbon (eg dichloromethane), an ether (eg tetrahydrofuran or dimethoxyethane) or acetonitrile, and in the presence of a base.

Compounds of formula (I) may be converted into other compounds of formula (I). Thus, for example, compounds of formula (I) wherein W is —S(O)ᵤ— and u is 1 or 2 may be prepared by oxidation of the corresponding compound of formula (I) wherein u is 0. The oxidation is preferably carried out using a peracid, eg peroxybenzoic acid, followed by treatment with a phosphine, such as triphenylphosphine. The reaction is suitably carried out in an organic solvent such as methylene chloride. Compounds of formula (I) wherein, for example, W is —N(R³⁵)— and R³⁵ is C₁₋₄alkyl can be prepared from compounds wherein R³⁵ is hydrogen by reductive alkylation. Compounds of formula (I) wherein Y is —N(R³⁵)— and R³⁵ is acetyl or benzoyl can be prepared from compounds wherein R³⁵ is hydrogen by acylation.

Compounds of formula (II), (IIA) and (IIB), wherein A is —C(O)—, —N(R⁷)—CH₂— or —CH(NR⁸R⁹)—, R⁴ or R⁵ are hydroxy or R⁴ and R⁵ taken together with the intervening atoms form a cyclic group having the following structure:

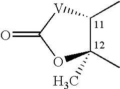

wherein V is a bivalent radical selected from —O— and —N(R¹⁷)—, and R³ is C₁₋₄alkyl, or C₃₋₆alkenyl optionally substituted by 9- or 10-membered fused bicyclic heteroaryl are known compounds or they may be prepared by analogous methods to those known in the art. Thus they can be prepared according to the procedures described in EP 0 307 177, EP 0 248 279, WO 00/78773 and WO 97/42204.

Compounds of formula (II), (IIA), (IIB) and (IIC) wherein A is —N(CH₃)CH₂—, R⁴ or R⁵ are hydroxy or R⁴ and R⁵ taken together with the intervening atoms form a cyclic group having the following structure:

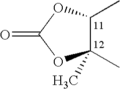

and R⁸ is hydrogen are known compounds or they may be prepared by analogous methods to those known in the art. Thus they can be prepared according to the procedures described in EP 0 508 699, J. Chem. Res. Synop. (1988, pages 152-153) and U.S. Pat. No. 6,262,030.

Compounds of formula (II), (IIA) and (IIB), wherein A is —C(═NR¹⁰)—, R⁴ or R⁵ are hydroxy or R⁴ and R⁵ taken together with the intervening atoms form a cyclic group having the following structure:

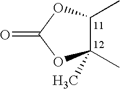

and R⁸ is hydrogen, are known compounds or they may be prepared by analogous methods to those known in the art. Thus they can be prepared according to the procedures described in EP 0 284 203.

Compounds of formula (II), (IIA), (IIB) and (IIC) wherein A is —C(O)—, R⁴ and R⁵ taken together with the intervening atoms form a cyclic group having the following structure:

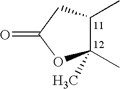

R⁶ is hydrogen and R³ is C₁₋₄alkyl may be prepared by decarboxylation of a compound of formula (XIII), wherein R³⁷ is a hydroxy protecting group followed, if required, by removal of the protecting group R² or R³⁷.

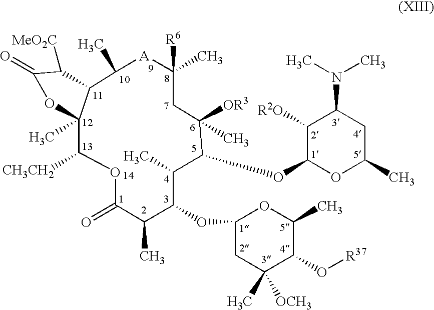

The decarboxylation may be carried out in the presence of a lithium salt such as lithium chloride, preferably in an organic solvent such as dimethylsulfoxide.

Compounds of formula (II), (IIA), (IIB) and (IIC) wherein A is —C(O)—, R⁴ and R⁵ taken together with the intervening atoms form a cyclic group having the following structure:

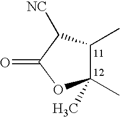

and R³ is C₁₋₄alkyl may be prepared according to the procedures described in WO 02/50091 and WO 02/50092.

Compounds of formula (III) and (IIIA) wherein U¹ is —W(CH₂)ᵣN(R³⁵)— or —W(CH₂)ᵣ—, wherein W is —N(R³⁵)—, —O— or —S—, may be prepared by reaction of HU^{1z}R^{14z} (VIII), wherein U^{1z} has the meaning defined above with R³⁸OC(O)(CH₂)_{d}L (XIV) wherein R³⁸ is carboxyl protecting group and L is a suitable leaving group, followed by removal of R³⁸. Suitable R³⁸ carboxyl protecting groups include tert-butyl, allyl or benzyl.

Compounds of formula (III) and (IIIA) may also be prepared by reaction of HU^{1z}R^{14z} (VIII) with acrylonitrile followed by hydrolysis of the nitrile to the acid, or by reaction of HU^{1z}R^{14z} (VIII) with tert-butyl acrylate followed by removal of the tert-butyl group.

Compounds of formula (VIII) wherein U¹ is —W(CH₂)ᵣX— in which X is —N(R³⁵)—, —O— or —S—, may be prepared by reaction of a compound of formula R^{14z}L (XV), wherein L is a suitable leaving group such as chlorine, fluorine or bromine, with a compound of formula —W(CH₂)ᵣX— (XVI) in which X is —N(R³⁵)—, —O— or —S—.

Compounds of formula (I) wherein R¹³ is —O(CH₂)ₐU¹R¹⁴, U¹ is —W(CH₂)ᵣX— or —W(CH₂)ᵣ, and W is —C(O)N(R³⁵)—, may be prepared by reaction of compounds of formula (XVII)

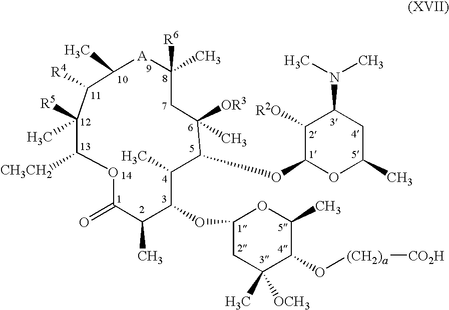

with a suitable amine compound.

All publications, including but not limited to patents and patent applications, cited in this specification are herein incorporated by reference as if each individual publication were specifically and individually indicated to be incorporated by reference herein as though fully set forth.

In order that the invention may be more fully understood the following examples are given by way of illustration only.

### EXAMPLES

The following abbreviations are used in the text: 9-BBN for 9-borabicyclo[3.3.1]nonane, BOC for tert-butoxycarbonyl, DBU for 1,8-diazabicyclo[5.4.0]undec-7-ene, DCM for dichloromethane, DMF for N,N-dimethylformamide, DMSO for dimethyl sulfoxide, EtOAc for ethyl acetate, EtOH for ethanol, MeCN for acetonitrile, MeOH for methanol, TFA for trifluoroacetic acid, THF for tetrahydrofuran, MgSO₄ for anhydrous magnesium sulphate, Na₂SO₄ for anhydrous sodium sulphate, HPLC for high performance liquid chromatography, ee for enantiomeric excess, and SCX chromatography for strong cation exchange chromatography.

### Examples

2′-O-Acetyl-6-O-methyl-erythromycin A may be prepared by the procedure described by W. R. Baker et al. in J. Org. Chem. 1988, 53, 2340 and 2′-O-acetyl-azithromycin-11,12-carbonate may be prepared by the procedure described by S. Djokic et al. in J. Chem. Res. (S) 1988, 152.

Erythromycin A (9E)-oxime may be prepared by the procedure described by R. R. Wilkening in EP 0 508 726 A1.

Erythromycin A (9E) methoxime may be prepared by the procedure described by J. R. Everett et al. in J. Chem. Soc. Perkin 2, 1989, 11, 1719-1728.

6-O-Methyl erythromycin A (9E)-oxime may be prepared by the procedure described by R. A. Dominguez et al in US 2003023053.

2′-O-acetyl-azithromycin and 2′-O-Acetyl-azithromycin-11,12-carbonate may be prepared by the procedures described by S. Djokic et al. in J. Chem. Res. (S) 1988, 152.

2′-O-Acetyl-erythromycin A-(9E)-O-acetyl-oxime may be prepared by the procedure described by J Berge et. al. in WO 2004039822.

Erythromycin A-(9E)-O-methoxymethyloxime may be prepared by the procedure described by Gasc, Jean Claude et al. in Journal of Antibiotics., 1991, 44(3), 313-30.

Erythromycin A (9E)-O-(1-methoxy-1-methylethyl)-oxime may be prepared by the procedure described by S. Morimoto et al. in U.S. Pat. No. 4,990,602.

Erythromycin A (9E)-O-(2-diethylaminoethyl)-oxime may be prepared by the procedure described by S. Gouin d'Ambrieres et al. in U.S. Pat. No. 4,349,545.

Where a TFA containing eluent is indicated, reverse phase HPLC refers to the use of an XTerra MS C18 column with a gradient of MeCN containing 0.1% TFA in water containing 0.1% TFA as eluent.

### Intermediate 1

### 9-[(2-Aminoethyl)thio]-5-(R,S)-methyl-1-oxo-6,7-dihydro-1H,5H-pyrido[3,2,1-ij]quinoline-2-carboxylic Acid Trifluoroacetate Salt

### a) Ethyl 9-fluoro-5-(R,S)-methyl-1-oxo-6,7-dihydro-1H,5H-pyrido[3,2,1-ij]quinoline-2-carboxylate

A suspension of flumequine (1.045 g, 4 mmol), iodoethane (3.1 mL, 40 mmol) and potassium carbonate (2.75 g, 20 mmol) in dimethylformamide (50 mL) was heated at 70° C. for 4 h. The reaction mixture was concentrated in vacuo. The residue was pre-absorbed on silica gel and purified by chromatography (silica gel, 0 to 5% [9:1 methanol/20 M ammonia] in dichloromethane) to give the title compound as a white solid (1.1 g); ESMS m/z 290.3 [M+H]⁺.

### b) Ethyl 9-(2-tert-Butoxycarbonylaminoethylthio)-5-(R,S)-methyl-1-oxo-6,7-dihydro-1H,5H-pyrido[3,2,1-ij]quinoline-2-carboxylate

A suspension of Intermediate 1a (1.1 g, 3.8 mmol), tert-butyl N-(2-mercaptoethyl)-carbamate (2.89 mL, 17.1 mmol) and potassium carbonate (2.36 g, 17.1 mmol) in dimethylsulfoxide (60 mL) was heated at 100° C. After 24 h the reaction mixture was cooled, filtered and partitioned between water/dichloromethane. The organic phase was separated, dried and evaporated. The residue was purified by chromatography (silica gel, 100% hexane-100% dichloromethane then 0-3% [9:1 methanol/20 M ammonia] in dichloromethane) to give the title compound (1.1 g); ESMS m/z 447.4 [M+H]⁺.

### c) Ethyl 9-(2-Aminoethylthio)-5-(R,S)-methyl-1-oxo-6,7-dihydro-1H,5H-pyrido[3,2,1-ij]quinoline-2-carboxylate Trifluoroacetate Salt

A solution of Intermediate 1b (1.7 g, 3.8 mmol) in dichloromethane (100 mL) was treated with trifluoroacetic acid (6 mL) and the reaction stirred at room temperature for 1 h. The solution was evaporated and the residue was partitioned between water and dichloromethane. The combined organic extracts were dried (MgSO₄), concentrated in vacuo to give the title compound (0.92 g) as a beige solid; ESMS m/z 347.3 [M+H]⁺.

### d) 9-(2-Aminoethylthio)-5-(R,S)-methyl-1-oxo-6,7-dihydro-1H,5H-pyrido[3,2,1-ij]quinoline-2-carboxylic Acid Trifluoroacetate Salt

A suspension of Intermediate 1c (0.92 g, 2.65 mmol) in 1,4-dioxan (20 mL) was ultrasonicated then treated with 2M sodium hydroxide (1.46 mL). The reaction was stirred for 17 h at room temperature. Solid carbon dioxide was added and the reaction mixture was concentrated. The residue was treated with dichloromethane/trifluoroacetic acid (3 mL/3 mL) and purified by reverse phase HPLC to give the title compound (0.615 g) as a white solid; ESMS m/z 319.3 [M+H]⁺.

### Intermediate 2

### 9-[(2-Aminoethyl)thio]-5-(*)-methyl-1-oxo-6,7-dihydro-1H,5H-pyrido[3,2,1-ij]quinoline-2-carboxylic Acid Hydrochloride Salt

### a) Ethyl 9-(2-tert-Butoxycarbonylaminoethylthio)-5-(*)-methyl-1-oxo-6,7-dihydro-1H,5H-pyrido[3,2,1-ij]quinoline-2-carboxylate

Normal phase chiral hplc over cellulose eluting with hexane/ethyl acetate (70:30) of Intermediate 1b (0.3 g, 0.67 mmol) gave the “less polar isomer” (0.1 g, 0.22 mmol), retention time 7 min, ee≧99%.

### b) 9-[(2-Aminoethyl)thio]-5-(*)-methyl-1-oxo-6,7-dihydro-1H,5H-pyrido[3,2,1-ij]quinoline-2-carboxylic Acid Hydrochloride Salt

A mixture of Intermediate 2a (0.1 g, 0.22 mmol) in dioxane (4 mL) and 2M hydrochloric acid (4 mL) was heated at 50° C. After 14 h the mixture was cooled and the solvent evaporated to yield the title compound (100%). Chiral HPLC indicated an ee≧98%; ESMS m/z 319.3 [M+H]⁺.

### Intermediate 3

### 9-[(2-Aminoethyl)thio]-5-(*)-methyl-1-oxo-6,7-dihydro-1H,5H-pyrido[3,2,1-ij]quinoline-2-carboxylic Acid Hydrochloride Salt

### a) Ethyl 9-(2-tert-Butoxycarbonylaminoethylthio)-5-(*)-methyl-1-oxo-6,7-dihydro-1H,5H-pyrido[3,2,1-ij]quinoline-2-carboxylate

Normal phase chiral HPLC over cellulose eluting with hexane/ethyl acetate (70:30) of Intermediate 1b (0.3 g, 0.67 mmol) gave the “more polar isomer” (0.1 g, 0.22 mmol), retention time 8.3 min, ee≧99%.

### b) 9-[(2-Aminoethyl)thio]-5-(*)-methyl-1-oxo-6,7-dihydro-1H,5H-pyrido[3,2,1-ij]quinoline-2-carboxylic Acid Hydrochloride Salt

A mixture of Intermediate 2a (0.1 g, 0.22 mmol) in dioxane (4 mL) and 2M hydrochloric acid (4 mL) was heated at 50° C. After 14 h the mixture was cooled and the solvent evaporated to yield the title compound (100%). Chiral HPLC indicated an ee≧98%; ESMS m/z 319.3 [M+H]⁺.

### Intermediate 4

### 9-[(2-Aminoethyl)sulfonyl]-5-(R,S)-methyl-1-oxo-6,7-dihydro-1H,5H-pyrido[3,2,1-ij]quinoline-2-carboxylic Acid Trifluoroacetate Salt

### a) Ethyl 9-{[2-({[(1,1-dimethylethyl)oxy]carbonyl}amino)ethyl]sulfonyl}-5-(R,S)-methyl-1-oxo-6,7-dihydro-1H,5H-pyrido[3,2,1-ij]quinoline-2-carboxylate

A solution of Intermediate 1a (0.5 g, 1.12 mmol) in dichloromethane (25 mL) at 5° C. was treated with 77% 3-chloroperoxybenzoic acid (0.5 g, 2.24 mmol). After 2 h a further portion (0.12 g, 0.53 mmol) of oxidising agent was added and stirring continued for 1 h. The mixture was quenched with 10% aqueous sodium metabisulfite solution (20 mL), the organic layer separated and washed with sodium bicarbonate solution. The organic phase was dried and evaporated to give the crude compound. Purification by chromatography (silica gel, 0 to 3% [9:1 methanol/20 M ammonia] in dichloromethane) gave the title compound (90%); ESMS m/z 479.3 [M+H]⁺.

### b) 9-{[2-({[(1,1-Dimethylethyl)oxy]carbonyl}amino)ethyl]sulfonyl}-5-(R,S)-methyl-1-oxo-6,7-dihydro-1H,5H-pyrido[3,2,1-ij]quinoline-2-carboxylic Acid Sodium Salt

A solution of Intermediate 4a (0.375 g, 0.78 mmol) in THF (6 mL) was treated with 2M sodium hydroxide (0.63 mL, 1.26 mmol). After stirring at 60° C. for 24 h the mixture was cooled and solid carbon dioxide added. The solvent was evaporated to yield the title compound (100%); ESMS m/z 451.3 [M+H]⁺.

### c) 9-(2-Aminoethylthio)-5-(R,S)-methyl-1-oxo-6,7-dihydro-1H,5H-pyrido[3,2,1-ij]quinoline-2-carboxylic Acid Trifluoroacetate Salt

A solution of Intermediate 4b (0.37 g, 0.78 mmol) in dichloromethane (3 mL) was treated with trifluoroacetic acid (3 mL) and the reaction stirred at room temperature for 0.5 h. The solution was evaporated to give the title compound (100%); ESMS m/z 351.2 [M+H]⁺.

### Intermediate 5

### 4″-O-Propenoylerythromycin A (9E)-oxime

### a) 2′-O-Acetylerythromycin A (9E)-acetyloxime

A solution of erythromycin A (9E)-oxime (8.5 g, 1.3) in dichloromethane (130 mL) was treated with sodium bicarbonate (2.09 g) followed by acetic anhydride (2.35 mL). After stirring overnight at room temperature the mixture was diluted with dichloromethane and washed with water. The organic layer was separated, dried and evaporated. The crude product was taken up in ethyl acetate and rewashed with saturated aqueous sodium bicarbonate. The organic layer was separated, dried and evaporated to yield the title compound as a solid. ESMS m/z 833.9 [M+H⁺].

### b) 2′-O-Acetyl-4″-O-propenoylerythromycin A (9E)-acetyloxime

A mixture of Intermediate 5a (8g), triethylamine (4 mL) and 3-chloropropionyl chloride (1.37 mL) in toluene (200 mL) was stirred at 20° C. for 20 h. The reaction mixture was concentrated by evaporation under reduced pressure then partitioned between a saturated solution of NH₄Cl and ethyl acetate. The organic phase was dried over Na₂SO₄, filtered and evaporated under reduced pressure. The crude product was purified by flash-chromatography 0-10% (9:1 MeOH/20M NH₃) in dichloromethane affording the title compound (4.0 g); ESMS m/z 887.6 [M+H]⁺.

### c) 4″-O-Propenoylerythromycin A (9E)-oxime

Intermediate 5b (4.0 g) was dissolved in MeOH (200 mL) and stirred at 55° C. for 20 h, then at room temperature for 72 h. The solvent was evaporated under reduced pressure affording the title compound (3.53 g); ESMS m/z 803.5 [M+H]⁺.

### Intermediate 6

### 4″-O-Propenoylerythromycin A (9E)-methoxime

### a) 2′-O-Acetylerythromycin A (9E)-methoxime

A solution of erythromycin A (9E) methoxime (5.7 g) in dichloromethane (70 mL) was treated with triethylamine (2.25 mL) followed by acetic anhydride (1.18 mL). After stirring overnight at room temperature the mixture was diluted with dichloromethane and washed with aqueous sodium bicarbonate. The organic layer was separated, dried and evaporated to yield the title compound as a solid; ESMS m/z 805.8 [M+H⁺].

### b) 2′-O-Acetyl-4″-O-propenoylerythromycin A (9E)-methoxime

Using a similar procedure to that described in Example 5b, Intermediate 6a (5.3 g) gave the title compound as a white solid. ESMS m/z 859.8 [M+H⁺].

### c) 4″-O-Propenoylerythromycin A (9E)-methoxime

Using a similar procedure to that described in Example 5c, Intermediate 6b (4.17 g) gave the title compound as a white solid; ESMS m/z 817.6 [M+H⁺].

### Intermediate 7

### 4″-O-Propenoyl-6-O-methylerythromycin A

### a) 2′-O-Acetyl-4″-O-propenoyl-6-O-methylerythromycin A

To a solution of 2′-O-acetyl-6-O-methyl-erythromycin A (1.1 g) in dichloromethane (20 mL) pyridine (1.7 mL) and acryloyl chloride (1.1 mL) were added at 0° C. After 2 h a further addition of pyridine (1.7 mL) and of acryloyl chloride (1.1 mL) was performed. The reaction mixture was quenched with a saturated solution of NH₄Cl (10 mL) and extracted with dichloromethane (3×20 mL). The organic phase was washed with a saturated solution of NaHCO₃ (10 mL), water (10 mL), dried (Na₂SO₄), filtered and evaporated under reduced pressure. The crude product was purified by flash-chromatography, 0-10% (9:1 MeOH/20 M NH₃) in dichloromethane, affording the title compound (470 mg); ESMS m/z 844 [M+H]⁺.

### b) 4″-O-Propenoyl-6-O-methylerythromycin A

Intermediate 7a (1.82 g) was dissolved in MeOH (100 mL) and stirred at 60° C. for 4 h, then at room temperature for 16 h. The solvent was evaporated under reduced pressure and the crude product was purified by flash chromatography (eluent: MeOH/DCM/NH₄OH 5/90/0) affording the title compound; ESMS m/z 802 [M+H]⁺.

### Intermediate 8

### 4″-O-Propenoyl-azithromycin-11,12-carbonate

### a) 2′-O-Acetyl-4″-O-propenoyl-azithromycin-11,12-carbonate

A solution of 2′-O-acetyl-azithromycin-11,12-carbonate (10.9 g) in toluene (300 mL) was stirred at room temperature under argon atmosphere. To this solution triethylamine (12.66 mL) and 3-chloro-propionyl chloride (1.94 mL) were added in two portions over a period of 10 minutes. After 20 minutes the solution was diluted with a saturated aqueous solution of NaHCO₃ (300 mL) and extracted with toluene (4×80 mL). The collected organic phase was dried, filtered and concentrated under reduced pressure affording the title compound (11.0 g); ESMS m/z 872 [M+H]⁺.

### b) 4″-O-Propenoyl-azithromycin-11,12-carbonate

A solution of Intermediate 8a (11.0 g) in MeOH (200 mL) was stirred at room temperature for 48 h. The solvent was evaporated under reduced pressure affording the title compound (9.81 g); ESMS m/z 872 [M+H]⁺.

### Intermediate 9

### 4″-O-Propenoyl-azithromycin

To a solution of Intermediate 8b (1.3 g, 1.49 mmol) in acetonitrile (50 mL), a saturated aqueous solution of potassium carbonate (30 mL) was added at room temperature. The resulting mixture was heated to 70° C. for 8 h. The mixture was then diluted with water (100 mL), extracted with ethyl acetate (4×30 mL). The combined organic phases were dried, filtered and evaporated. The crude product was purified by chromatography over silica eluting with 0-10% (9:1 MeOH/20M NH₃) in dichlormethane to give the title compound as a white solid; ESMS m/z 872 [M+H]⁺.

### Intermediate 10

### 4″-O-Propenoylerythromycin A (9E)-methoxymethyloxime

### a) Erythromycin A (9E)-methoxymethyloxime

A solution of erythromycin A (9E)-oxime (10.11 g, 13.5 mmol) dissolved in dichloromethane (25 mL) at 5° C. was treated with tetra-n-butylammonium bromide (0.486 g) and 2M sodium hydroxide solution (25 mL). The resultant mixture was stirred vigorously and chloromethoxymethane (1.5 mL, 20 mmol), added in a single portion. After 0.75 h the mixture was quenched by the addition of brine (50 mL) and the organic material extracted with chloroform (2×150 mL). The combined organic phases were dried and evaporated to yield the crude product. Crystallisation from ethyl acetate gave the title compound as white solid; ESMS m/z 793.8 [M+H]⁺.

### b) 2′-O-Acetylerythromycin A (9E)-methoxymethyloxime

A suspension of Intermediate 10a (3.94 g, 4.96 mmol) in dichloromethane (20 mL) was treated with acetic anhydride (0.57 g, 5.58 mmol). After stirring for 48 h water (30 mL) was added and the pH of the mixture adjusted to 9-10 by the addition of 2M sodium hydroxide solution. The organic material was extracted with dichloromethane (2×50 mL) and the combined extracts were dried and evaporated to yield the title compound as a white solid; ESMS m/z 835.8 [M+H]⁺.

### c) 2′-O-Acetyl-4″-O-propenylerythromycin A (9E)-methoxymethyloxime

A solution of Intermediate 10b (3.8 g, 4.55 mmol) dissolved in toluene (50 mL) and triethylamine was treated with 3-chloropropionyl chloride (0.54 mL, 5.7 mmol). After stirring for 24 h the mixture was diluted with ethyl acetate (100 mL) and washed with water (2×50 mL). The organic phase was separated, dried and evaporated to give the crude product. Purification by chromatography over silica gel eluting with 0-10% (9:1 MeOH/20M NH₃) in dichloromethane to give the title compound as a white solid; ESMS m/z 889.9 [M+H]⁺.

### d) 4″-O-Propenylerythromycin A (9E)-methoxymethyloxime

A solution of Intermediate 10c (1.80 g, 2.02 mmol) dissolved in methanol (50 mL) was stirred at 50° C. for 16 h. The methanol was evaporated to yield the title compound (83%) as a white solid; ESMS m/z 877.7 [M+H]⁺.

### Intermediate 11

### 9-({2-[(2-aminoethyl)oxy]ethyl}amino)-5-(RS)-methyl-1-oxo-6,7-dihydro-1H,5H-pyrido[3,2,1-ij]quinoline-2-carboxylic Acid

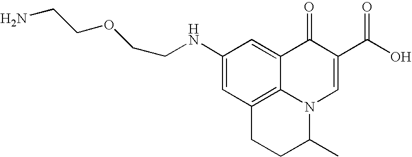

A solution of flumequine (0.13 g, 0.5 mmol) and {2-[(2-aminoethyl)oxy]ethyl}amine (0.25 mL) in N-methyl-2-pyrrolidinone (0.25 mL) was microwaved at 200° C. for 1 h. The reaction mixture was purified by preparative reverse phase HPLC (MeCN/H₂O/0.1% HCO₂H eluent) to give the title compound as a yellow oil (0.14 g). ESMS m/z 346.4 [M+H]⁺.

### Intermediate 12

### 9-[(2-aminoethyl)amino]-5-(R,S)-methyl-1-oxo-6,7-dihydro-1H,5H-pyrido[3,2,1-ij]quinoline-2-carboxylic Acid

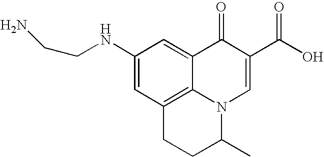

A solution of flumequine (0.26 g, 1 mmol) and 1,2-diaminoethane (0.334 mL) in N-methyl-2-pyrrolidinone (0.5 mL) was microwaved at 200° C. for 0.5 h. The reaction mixture was concentrated then purified by preparative reverse phase HPLC (MeCN/H₂O/0.1% HCO₂H eluent) to give the title compound as a pale yellow solid (0.15 g). ESMS m/z 302.3 [M+H]⁺.

### Intermediate 13

### 4″-O-(1-Imidazolyl-carbonyl)-6-O-methyl-erythromycin A

6-O-Methyl-erythromycin A (30 g, 40.1 mmol) in THF (100 mL) was treated portionwise with carbonyldiimidazole (16 g, 97 mmol) with ice bath cooling. After 1 h the cooling bath was removed. After a further 48 h, THF (100 mL) and water (200 mL) were added causing slow precipitation of the title compound, which was collected by filtration and dried to give the title compound (24.7 g). Extraction of the mother liquors with diethyl ether gave a further 8.5 g of material which was precipitated from THF solution with water to give a further portion of the title compound as a white solid (3.92 g, total 28.64 g); ESMS m/z 842 [M+H]⁺.

### Intermediate 14

### 9-{[2-(Ethylamino)ethyl]thio}-5-(R,S)-methyl-1-oxo-6,7-dihydro-1H,5H-pyrido[3,2,1-ij]quinoline-2-carboxylic Acid Ethyl Ester

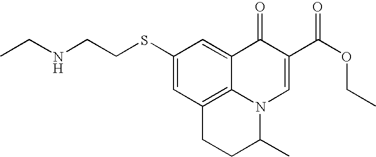

### a) 9{([2-tert-Butoxycarbonyl(ethylamino)ethyl]lthio}-5-(R,S)-methyl-1-oxo-6,7-dihydro-1H,5H-pyrido[3,2,1-ij]quinoline-2-carboxylic Acid Ethyl Ester

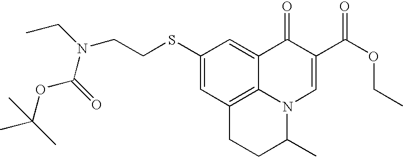

To a solution of Intermediate 1b (0.58 g, 1.25 mmol) in DMF (8 mL) was added a 60% suspension of sodium hydride (0.12 g). After 10 min iodoethane (0.28 mL, 3.5 mmol) was added and the reaction mixture stirred for 16 h. After concentration the residue was partitioned between 10% aqueous solution of citric acid and dichloromethane. The organic phase was washed with brine, dried and evaporated. The residue was purified by chromatography (silica gel, 0 to 2% [9:1 methanol/20 M ammonia] in dichloromethane) to give the title compound as a pale yellow solid (0.547 g); ESMS m/z 475.3 [M+H]⁺.

### b) 9-{[2-(Ethylamino)ethyl]thio}-5-(R,S)-methyl-1-oxo-6,7-dihydro-1H,5H-pyrido[3,2,1-ij]quinoline-2-carboxylic Acid Ethyl Ester

A solution of Intermediate 14a (0.547 g, 1.15 mmol) in dichloromethane (8 mL) was treated with TFA (3 mL). After 0.5 h the reaction mixture was concentrated and the residue partitioned between dichloromethane and 1 M aqueous potassium carbonate. The organic phase was dried, evaporated and the residue purified by chromatography (silica gel, 0 to 5% [9:1 methanol/20M ammonia] in dichloromethane) to give the title compound as a pale yellow solid (0.396 g); ESMS m/z 375.3 [M+H]⁺.

### Intermediate 15

### 9-[3-(2-Aminoethoxy)propyl]-5-(R,S)-methyl-1-oxo-6,7-dihydro-1H,5H-pyrido[3,2,1-ij]quinoline-2-carboxylic Acid Trifluoroacetic Acid Salt

### a) 5-(R,S)-Methyl-1-oxo-6,7-dihydro-1H,5H-pyrido[3,2,1-ij]quinoline-2-carboxylic Acid Ethyl Ester

2-(R,S)-Methyl-1,2,3,4-tetrahydro-quinoline (5.00 g) was mixed with 2-ethoxymethylene-malonic acid diethyl ester (8.10 g). The mixture was stirred at 130° C. under nitrogen for 2 h. Polyphosphoric acid (8.50 g) was added to this mixture and the resulting mixture was heated at 130° C. for 3 h under nitrogen. Water (50 mL) was added to the cooled mixture followed by extraction with ethyl acetate (3×50 mL). The organic phase was dried and evaporated in vacuo. The residue was purified by flash chromatography (silica gel, 0-2% methanol in dichloromethane) to give the title compound (4.40 g); ESMS m/z 272.0 [M+H]⁺.

### b) 9-Bromo-5-(R,S)-methyl-1-oxo-6,7-dihydro-1H,5H-pyrido[3,2,1-ij]quinoline-2-carboxylic Acid Ethyl Ester

Intermediate 15a (2.00 g) was dissolved in DMF (30 mL) and N-bromosuccinimide (2.60 g) was added at 0° C. The mixture was stirred at room temperature for 12 h. The white precipitate was filtered off and washed with diethyl ether to obtain the title compound (1.60 g); ESMS m/z 349.8 [M+H]⁺.

### c) 9-Bromo-5-(R,S)-methyl-1-oxo-6,7-dihydro-1H,5H-pyrido[3,2,1-ij]quinoline-2-carboxylic Acid

Intermediate 15b (2.00 g) was dissolved in dioxan (30 mL) and 2N aqueous NaOH (14 mL) was added. The solution was stirred at reflux for 3 h. The mixture was acidified with 20% aqueous citric acid and extracted with dichloromethane (3×30 mL). The organic phase was dried and evaporated in vacuo, to give the title compound (1.66 g); ESMS m/z 321.9 [M+H]⁺.

### d) (2-Prop-2-ynyloxy-ethyl)-carbamic Acid Tert-Butyl Ester

To a solution of (2-hydroxyethyl)carbamic acid tert-butyl ester (1.56 g) in dichloromethane (25 mL) benzyltrimethylammonium chloride (0.18 g) was added. To this solution aqueous 50% NaOH (30g) and propargyl bromide (1.05 mL of 80% solution in toluene) were added and the mixture was vigorously stirred at room temperature for 3 h. The phases were diluted, separated and the organic phase washed with water (2×15 mL), dried and finally evaporated in vacuo. The residue was purified by flash chromatography (silica gel, 0-5% methanol in dichloromethane) to give the title compound as a pale-orange oil (1.53 g); ESMS m/z 222.4 [M+Na]⁺.

### e) 9-[3-(2-tert-Butoxycarbonylamino-ethoxy)-prop-1-ynyl]-5-(R,S)-methyl-1-oxo-6,7-dihydro-1H,5H-pyrido[3,2,1-ij]quinoline-2-carboxylic Acid

Intermediate 15c (1.02 g) and copper (I) iodide (0.060 g) were suspended in a mixture of DMF (20 mL) and triethylamine (15 mL); the suspension was degassed by nitrogen bubbling. After 30 min Intermediate 15d (1.01 g) and dichlorobis(triphenylphosphine)palladium (II) (0.067 g) were added under nitrogen and the mixture was heated at 100° C. for 2 h. After this period dichloromethane (100 mL) was added and washed with aqueous 20% citric acid (2×50 mL). The organic phase was dried and evaporated in vacuo. The residue was purified by flash chromatography (silica gel, 0-3% methanol in dichloromethane) to give the title compound (1.73 g); ESMS m/z 440.9 [M+H]⁻.

### f) 9-[3-(2-tert-Butoxycarbonylaminoethoxy)propyl]-5-(R,S)-methyl-1-oxo-6,7-dihydro-1H,5H-pyrido[3,2,1-ij]quinoline-2-carboxylic Acid

Intermediate 15e (1.73 g) was dissolved in MeOH (40 mL) and wet (50% dispersion in water) 10% palladium on carbon (0.350g) was added. The mixture was hydrogenated at room temperature at 20 psi of hydrogen for 2 h. The catalyst was filtered off and the solvent evaporated in vacuo to give the title compound (1.70 g); ESMS m/z 445.0 [M+H]⁺.

### g) 9-[3-(2-Aminoethoxy)propyl]-5-(R,S)-methyl-1-oxo-6,7-dihydro-1H,5H-pyrido[3,2,1-ij]quinoline-2-carboxylic Acid Trifluoroacetic Acid Salt

A solution of Intermediate 15f (1.70 g) in dichloromethane (60 mL) was treated with TFA (4.5 mL) and stirred at room temperature for 2 h. The solvent was evaporated in vacuo; the residue was re-evaporated from dichloromethane (2×) and triturated with diisopropylether to give the title compound as a solid (1.41 g); ESMS m/z 345.0 [M+H]⁺.

### Intermediate 16

### 9-[2-(2-Aminoethoxy)ethylthio]-5-(R,S)-methyl-1-oxo-6,7-dihydro-1H,5H-pyrido[3,2,1-ij]quinoline-2-carboxylic Acid Trifluoroacetic Acid Salt

### a) 9-Iodo-5-(RS)-methyl-1-oxo-6,7-dihydro-1H,5H-pyrido[3,2,1-ij]quinoline-2-carboxylic Acid Ethyl Ester

Intermediate 15b (0.175 g), copper (I) iodide (0.009 g), sodium iodide (0.375 g) and N,N′-dimethylethylene-1,2-diamine (0.018 g) were dissolved in dry ethanol (2 mL). The mixture was heated at 130° C. under microwave irradiation in a sealed tube for 3 h. Water was added to the cooled mixture (10 mL) and the aqueous phase was extracted with dichloromethane (3×20 mL) The organic phase was dried and evaporated in vacuo to give the title compound (0.180g); ESMS m/z 397.8 [M+H]⁺.

### b) [2-(2-Hydroxyethoxy)ethyl]-carbamic Acid Tert-Butyl Ester

To a solution of 2-(2-aminoethoxy)ethanol (5.00 g) in water (35 mL) potassium hydroxide (2.93 g) was added at 0° C. To this mixture maintained at 0° C., a solution of di-tert-butyldicarbonate (11.40 g) in dioxan (17 mL) was added dropwise. The resulting mixture was stirred at 0° C. for 1 h and left at room temperature for 4 h. The dioxan was evaporated and the aqueous solution was extracted with dichloromethane (2×25 mL). The organic phase was dried and evaporated in vacuo. The residue was purified by flash chromatography (silica gel, ethyl acetate/petroleum ether 40/60 to 60/40) to give the title product (8.50 g); ESMS m/z 205.3 [M+H]⁺.

### c) Toluene-4-sulfonic acid 2-(2-tert-butoxycarbonylaminoethoxy)-ethyl Ester

Intermediate 16b (7.39 g) was dissolved in dichloromethane (75 mL) and to the solution triethylamine (5.00 mL) and p-toluenesulfonyl chloride (6.87 g) were added at 0° C. The resulting solution was stirred at room temperature overnight. The suspension was filtered off and the organic phase was evaporated in vacuo. The residue was purified by flash chromatography (silica gel, ethyl acetate/petroleum ether from 20/80 to 40/60) to give the title product (11.25 g); ESMS m/z 360.4 [M+H]⁺.

### d) Thioacetic acid S-[2-(2-tert-butoxycarbonylaminoethoxy)ethyl]ester

To a solution of Intermediate 16c (11.69 g) in acetone (250 mL) potassium thioacetate (7.42 g) was added; the resulting suspension was heated under reflux for 2 h. The solid was filtered and washed with acetone. The solution was evaporated in vacuo and the residue was purified by filtration on a short silica pad (ethyl acetate/petroleum ether 20/80) to obtain the title compound (6.85 g); ESMS m/z 264.4 [M+H]⁺.

### e) 9-[2-(2-tert-Butoxycarbonylaminoethoxy)ethylthio]-5-(R,S)-methyl-1-oxo-6,7-dihydro-1H,5H-pyrido[3,2,1-ij]quinoline-2-carboxylic Acid

A suspension of Intermediate 16a (0.40 g), Intermediate 16d (0.33 g), copper (I) iodide (0.019 g), potassium carbonate (0.59 g) and ethylene glycol (0.22 mL) in absolute ethanol (8 mL) was degassed by nitrogen bubbling for 30 min. The mixture was then heated in a sealed tube at 120° C. for 1 h under microwave irradiation. The mixture was acidified with 20% aqueous citric acid and then extracted with ethyl acetate. The organic phase was dried and evaporated in vacuo. The residue was purified by flash chromatography (silica gel, 0-10% methanol in dichloromethane) to give the title compound (0.30 g); ESMS m/z 463.0 [M+H]⁺.

### f) 9-[2-(2-Aminoethoxy)ethylthio]-5-(R,S)-methyl-1-oxo-6,7-dihydro-1H,5H-pyrido[3,2,1-ij]quinoline-2-carboxylic Acid Trifluoroacetic Acid Salt

A solution of Intermediate 16e (0.30 g) in dichloromethane (15 mL) was treated with TFA (3 mL) and stirred at room temperature for 2 h. The solvent was evaporated in vacuo; the residue was re-evaporated from dichloromethane (2×) and triturated with diisopropylether to give the title compound as a solid (0.28 g); ESMS m/z 362.9 [M+H]⁺.

### Intermediate 17

### 8-[3-(2-Aminoethoxy)propyl]-6-oxo-1,2-dihydro-6H-pyrrolo[3,2,1-ij]quinoline-5-carboxylic Acid Trifluoroacetic Acid Salt

### a) 1-(Trifluoroacetyl)-2,3-dihydro-1H-indole

To a solution of 2,3-dihydro-1H-indole (1.19 g) in dichloromethane (10 mL) triethylamine (1.7 mL) was added. The mixture was cooled at 0° C. and trifluoroacetic anhydride (1.55 mL) was added dropwise. After 30 min at 0° C. water (50 mL) was added and the resulting mixture was extracted with diethyl ether (3×50 mL). The organic phase was dried and evaporated in vacuo. The residue was purified by crystallization (diisopropyl ether/diethyl ether) to give the title compound (1.68 g); ESMS m/z 216.0 [M+H]⁺.

### b) 1-(Trifluoroacetyl)-5-iodo-2,3-dihydro-1H-indole

Intermediate 17a (0.200 g) was dissolved in 1,2-dichloroethane (10 mL) and iodine (0.192 g) was added. To this solution bis(trifluoroacetoxy)iodobenzene was added and the resulting solution was stirred at room temperature for 20 h. Aqueous saturated Na₂SO₃ (10 mL) was added and the mixture was extracted with diethyl ether (2×15 mL). The organic phase was dried and evaporated in vacuo. The residue was purified by flash chromatography (silica gel, hexane 0-10% in diethyl ether) to give the title compound (0.225 g); ESMS m/z 341.8 [M+H]⁺.

### c) 5-Iodo-2,3-dihydro-1H-indole

Intermediate 17b (0.225 g) was dissolved in a mixture of MeOH (10 mL) and THF (10 mL). To this mixture aqueous 10% NaOH (10 mL) was added at room temperature. After 5 min water (20 mL) was added and the mixture was extracted with diethyl ether (3×15 mL). The organic phase was dried and evaporated in vacuo to give the title compound (0.200g); ESMS m/z 245.9 [M+H]⁺.

### d) 8-Iodo-6-oxo-1,2-dihydro-6H-pyrrolo[3,2,1-ij]quinoline-5-carboxylic Acid Ethyl Ester

A mixture of Intermediate 17c (0.200 g) and 2-ethoxymethylene-malonic acid diethyl ester (0.262 g) was heated at 110° C. under nitrogen for 3 h. To this mixture polyphosphoric acid (0.200 g) was added and the resulting mixture was heated at 120° C. under nitrogen for 3 h. Water was added (5 mL) and the mixture was extracted with ethyl acetate (3×5 mL). The organic phase was washed with brine (10 mL), dried and evaporated in vacuo. The residue was purified by flash chromatography (silica gel, 0-2% methanol in dichloromethane) to give the title compound (0.120g); ESMS m/z 369.7 [M+H]⁺.

### e) 8-[3-(2-tert-Butoxycarbonylamino-ethoxy)-prop-1-ynyl]-6-oxo-1,2-dihydro-6H-pyrrolo[3,2,1-ij]quinoline-5-carboxylic Acid Ethyl Ester

Intermediate 17d (0.184 g) and copper (I) iodide (0.003 g) were suspended in a mixture of CH₃CN (6 mL) and triethylamine (2.5 mL); the suspension was degassed bubbling nitrogen for 30 min. Intermediate 15d (0.149 g) and dichlorobis(triphenylphosphine)palladium (II) (0.012 g) were added under nitrogen and the mixture was heated at reflux for 2 h. After this period solvents were removed and dichloromethane (10 mL) was added and washed with aqueous 20% citric acid (2×10 mL). The organic phase was dried and evaporated in vacuo. The residue was purified by flash chromatography (silica gel, 0-3% methanol in dichloromethane) to give a brown solid that was triturated with MeOH to give the title compound (0.195 g); ESMS m/z 440.8 [M+H]⁺.

### f) 8-[3-(2-tert-Butoxycarbonylaminoethoxy)propyl]-6-oxo-1,2-dihydro-6H-pyrrolo[3,2,1-ij]quinoline-5-carboxylic Acid Ethyl Ester

Intermediate 17e (0.660 g) was dissolved in MeOH (15 mL) and wet (50% dispersion in water) 10% palladium on carbon (0.132 g) was added. The mixture was hydrogenated at room temperature at 20 psi for 2 h. The catalyst was removed by filtration and the solvent evaporated in vacuo to give the title compound (0.660 g); ESMS m/z 444.9 [M+H]⁺.

### g) 8-[3-(2-tert-Butoxycarbonylamino-ethoxy)-propyl]-6-oxo-1,2-dihydro-6H-pyrrolo[3,2,1-ij]quinoline-5-carboxylic Acid

To a solution of Intermediate 17f (1.41 g) in dioxan (30 mL) 2N NaOH (8 mL) was added. The resulting mixture was heated at reflux for 3 h. The mixture was acidified with 20% aqueous citric acid and extracted with dichloromethane (3×30 mL). The organic phase was dried and evaporated in vacuo, to give the title compound (1.32 g); ESMS m/z 417.0 [M+H]⁺.

### h) 8-[3-(2-Aminoethoxy)propyl]-6-oxo-1,2-dihydro-6H-pyrrolo[3,2,1-ij]quinoline-5-carboxylic Acid Trifluoroacetic Acid Salt

A solution of Intermediate 17g (1.32 g) in dichloromethane (65 mL) was treated with TFA (4 mL) and stirred at room temperature for 2 h. The solvent was evaporated in vacuo; the residue was re-evaporated from dichloromethane (2×) and triturated with diisopropylether to give the title compound as a solid (1.16 g); ESMS m/z 317.0 [M+H]⁺.

### Intermediate 18

### 8-[2-(2-Aminoethoxy)ethylthio]-6-oxo-1,2-dihydro-6H-pyrrolo[3,2,1-ij]quinoline-5-carboxylic Acid

### a) 8-[2-(2-tert-Butoxycarbonylaminoethoxy)ethylthio]-6-oxo-1,2-dihydro-6H-pyrrolo[3,2,1-ij]quinoline-5-carboxylic Acid

A suspension of Intermediate 17d (0.184 g), Intermediate 16d (0.164 g), copper (I) iodide (0.010 g), potassium carbonate (0.276 g) and ethylene glycol (0.125 mL) in absolute ethanol (4 mL) was degassed by nitrogen bubbling for 30 min. The mixture was then heated in a sealed tube at 130° C. for 1 h under microwave irradiation. The cooled mixture was acidified with 20% aqueous citric acid and then extracted with dichloromethane. The organic phase was dried and evaporated in vacuo to give the title compound (0.164 g); ESMS m/z 434.8 [M+H]⁺.

### b) 8-[2-(2-Aminoethoxy)ethylthio]-6-oxo-1,2-dihydro-6H-pyrrolo[3,2,1-ij]quinoline-5-carboxylic Acid

A solution of Intermediate 18a (1.07 g) in dichloromethane (50 mL) was treated with TFA (3 mL) and stirred at room temperature for 2 h. The solvent was evaporated in vacuo; the residue was re-evaporated from dichloromethane (2×) and triturated with diisopropylether; the solid residue was purified by SCX chromatography (elution with 100 mL of methanol and then with 200 mL of 20% 20M ammonia in methanol); the ammonia fraction was evaporated in vacuo to give the title compound as a solid (0.96 g); ESMS m/z 334.9 [M+H]⁺.

### Intermediate 19

### 2′-O-Acetyl-4″-O-(imidazol-1-yl-carbonyl)-erythromycin A-9(E)-O-methoxymethyloxime

### a) 2′-O-Acetylerythromycin A-9(E)-O-methoxymethyloxime

To a solution of erythromycin A-9(E)-O-methoxymethyloxime (4.05 g) in dichloromethane (50 mL) NaHCO₃ (0.60 g) was added followed by acetic anhydride (0.68 mL). After stirring overnight at room temperature the mixture was diluted with dichloromethane and washed with 5% aqueous NaHCO₃. The organic layer was separated, dried and evaporated to yield the title product (4.1 g) as a solid; ESMS m/z 835.8 [M+H]⁺.

### b) 2′-O-Acetyl-4″-O-(imidazol-1-yl-carbonyl)-erythromycin A-9(E)-O-methoxymethyloxime

To a solution of Intermediate 17a (1.10 g) in dry THF (40 mL) 4-dimethylaminopyridine (0.80 g) and carbonyl diimidazole (1.10 g) were added; the resulting mixture was stirred at room temperature for 6 h under nitrogen. To the solution ethyl acetate and 5% aqueous KH₂PO₄ were added (100 mL each) and the phases were separated. The organic phase was dried and evaporated in vacuo to give the title compound (1.10 g) as white foam; ESMS m/z 929.9 [M+H]⁺.

### Intermediate 20

### 2′-O-Acetyl-4″-O-(imidazol-1-yl-carbonyl)-erythromycin A-9(E)-O-(1-methoxy-1-methylethyl)-oxime

### a) 2′-O-Acetyl-erythromycin A-9(E)-O-(1-methoxy-1-methylethyl)-oxime

To a solution of erythromycin A-9(E)-O-(1-methoxy-1-methylethyl)-oxime (0.86 g) in dichloromethane (15 mL) NaHCO₃ (0.13 g) was added followed by acetic anhydride (0.15 mL). After stirring overnight at room temperature the mixture was diluted with dichloromethane and washed with 5% aqueous NaHCO₃. The organic layer was separated, dried and evaporated to yield the title product (0.90 g) as a solid; ESMS m/z 863.4 [M+H]⁺.

### b) 2′-O-Acetyl-4″-O-(imidazol-1-yl-carbonyl)-erythromycin A-9(E)-O-(1-methoxy-1-methylethyl)-oxime

To a solution of Intermediate 20a (0.90 g) in dry THF (25 mL) 4-dimethylaminopyridine (0.70 g) and carbonyl diimidazole (0.90 g) were added. The resulting mixture was stirred under nitrogen at room temperature for 6 h. To the solution ethyl acetate and 5% aqueous KH₂PO₄ were added (100 mL each) and the phases were separated. The organic phase was dried and evaporated in vacuo to give the title compound (0.95 g) as white foam; ESMS m/z 957.4 [M+H]⁺.

### Intermediate 21

### 2′-O-Acetyl-4″-O-(imidazol-1-yl-carbonyl)-azithromycin

### a) 2′-O-Acetyl-azithromycin

To a solution of azithromycin (2.00 g) in dichloromethane (25 mL) NaHCO₃ (0.29 g) was added followed by acetic anhydride (0.28 mL). After stirring overnight at room temperature the mixture was diluted with dichloromethane and washed with 5% aqueous NaHCO₃. The organic layer was separated, dried and evaporated to yield the title product (2.00 g) as a solid; ESMS m/z 791.2 [M+H].

### b) 2′-O-Acetyl-4″-O-(imidazol-1-yl-carbonyl)-azithromycin

Intermediate 21a (1.50 g) was dissolved in dry toluene (20 mL); triethylamine (0.61 mL) and carbonyl diimidazole (0.34 g) were added. The resulting solution was stirred at room temperature under nitrogen for 24 h. After this period a second portion of carbonyl diimidazole (0.34 g) was added and the solution stirred at room temperature for additional 24h. The solution was extracted with 5% aqueous NaHCO₃ (20 mL) dried and evaporated in vacuo to give the title compound (1.60 g); ESMS m/z 885.4 [M+H]⁺.

### Intermediate 22

### 2′-O-Acetyl-4″-O-(imidazol-1-yl-carbonyl)-erythromycin A-9(E)-O-cyanomethyloxime

### a) Erythromycin A-9(E)-O-cyanomethyloxime

To a solution of erythromycin A-9(E)-oxime (3.00 g) in dry THF (120 mL) a solution of tetrabutylammonium hydroxide 1 M in methanol (4.4 mL) was added at room temperature. After 5 min chloroacetonitrile (0.38 mL) was added dropwise and the resulting mixture was heated at 50° C. for 1 h. The solvent was removed in vacuo and the residue was dissolved in diethyl ether and extracted with brine (3×75 mL). The organic phase was dried and evaporated in vacuo. The residue was purified by flash chromatography (silica gel, 0-3% of 9/11 methanol/20M ammonia in dichloromethane) to give the title compound (2.27 g) as a solid; ESMS m/z 788.4 [M+H]⁺.

### b) 2′-O-Acetyl-erythromycin A-9(E)-O-cyanomethyloxime

To a solution of Intermediate 22a (2.24 g) in dichloromethane (50 mL) NaHCO₃ (0.36 g) was added followed by acetic anhydride (0.40 mL). After stirring overnight at room temperature the mixture was diluted with dichloromethane and washed with 5% aqueous NaHCO₃. The organic layer was separated, dried and evaporated to yield the title product (2.35 g) as a solid; ESMS m/z 830.4 [M+H]⁺.

### c) 2′-O-Acetyl-4″-O-(imidazol-1-yl-carbonyl)-erythromycin A-9(E)-O-cyanomethyloxime

To a solution of Intermediate 22b (2.35 g) in dry THF (90 mL) 4-dimethylaminopyridine (1.73 g) and carbonyl diimidazole (2.30 g) were added; the resulting mixture was stirred under nitrogen at room temperature for 6 h. The solvent was removed in vacuo and the residue was dissolved in ethyl acetate (150 mL) and extracted with 5% aqueous KH₂PO₄ (2×100 mL) and brine (100 mL). The organic phase was dried and evaporated in vacuo to give the title compound (2.65 g) as white foam; ESMS m/z 924.4 [M+H]⁺.

Michael Reaction—General Procedure

Typically a solution of one of the Intermediates 1-4 (0.3 mmol) and one of the Intermediates 5-10 (0.15 mmol) in dimethyl sulfoxide (1.5 mL), triethylamine (0.15 mL) was heated at 80° C. for 8-24 h. The mixture was cooled and the resultant solution subjected to mass-directed autoprep purification (eluent acetonitrile/water/formic acid) to yield the Michael adduct as the formate salt. The free base was prepared by chromatography over silica gel eluting with 0-10% (9:1 MeOH/20M ammonia) in dichloromethane.

| | Inter- | | | |
|---|---|---|---|---|
| | medi- | | | |
| Eg. | ates | Compound Name | Structure | m/e |
| 1 | 1 + 7 | 4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-6-O-methylerythromycin A | 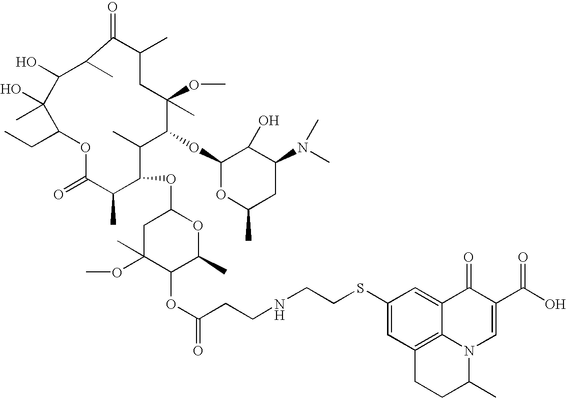 | 1120.9 |
| 2 | 1 + 9 | 4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-azithromycin | 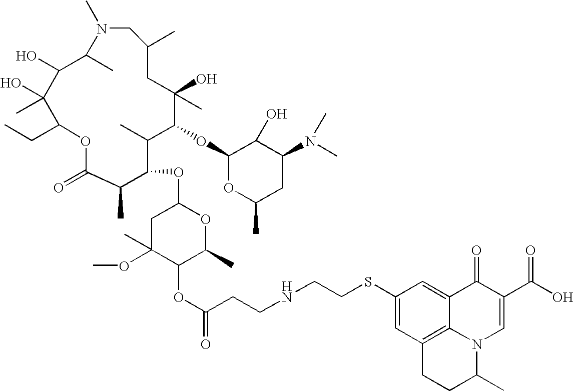 | 1122.0 |
| 3 | 1 + 8 | 4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-azithromycin-11,12-carbonate | 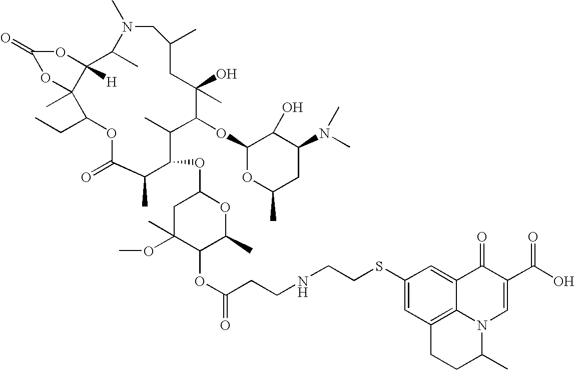 | 1148.0 |
| 4 | 1 + 5 | 4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-erythromycin A (9E)-oxime | 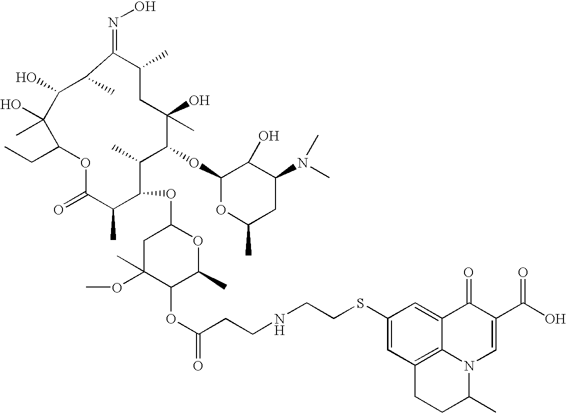 | 1122.0 |
| 5 | 1 + 10 | 4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-erythromycin A (9E)-methoxymethyloxime | 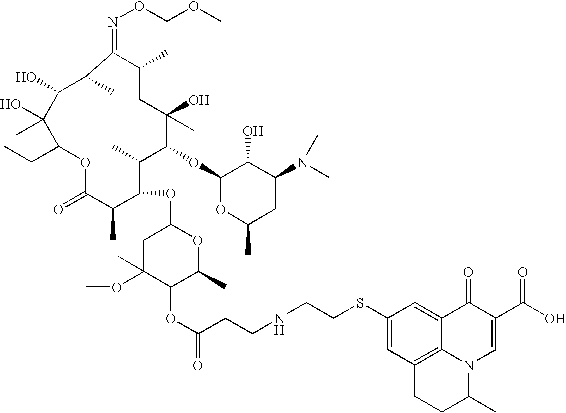 | 1165.8 |
| 6 | 4 + 7 | 4″-O-{[2-({2-[(2-carboxy-5-(R,S)-methyl-1-oxo-6,7-dihydro-1H,5H-pyrido[3,2,1-ij]quinolin-9-yl)sulfonyl]ethyl}amino)ethyl]propionyl}-6-O-methylerythromycin A | 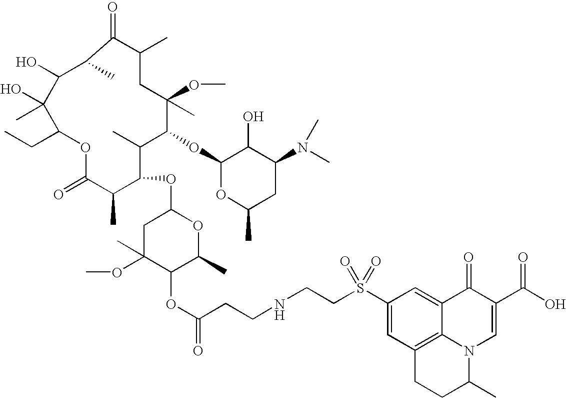 | 1153.1 |
| 7 | 4 + 9 | 4″-O-{[2-({2-[(2-carboxy-5-(R,S)-methyl-1-oxo-6,7-dihydro-1H,5H-pyrido[3,2,1-ij]quinolin-9-yl)sulfonyl]ethyl}amino)ethyl]propionyl}-azithromycin | 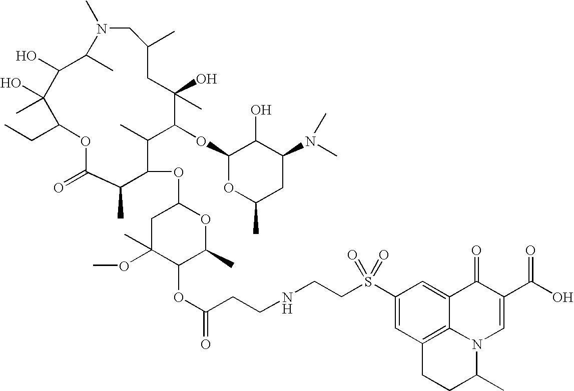 | 1154.0 |
| 8 | 4 + 6 | 4″-O-{[2-({2-[(2-carboxy-5-(R,S)-methyl-1-oxo-6,7-dihydro-1H,5H-pyrido[3,2,1-ij]quinolin-9-yl)sulfonyl]ethyl}amino)ethyl]propionyl}-erythromycin A (9E)-methoxime | 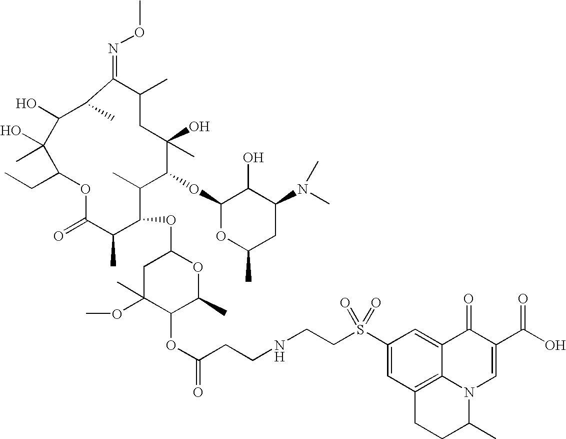 | 1168.2 |
| 9 | 4 + 10 | 4″-O-{[2-({2-[(2-carboxy-5-(R,S)-methyl-1-oxo-6,7-dihydro-1H,5H-pyrido[3,2,1-ij]quinolin-9-yl)sulfonyl]ethyl}amino)ethyl]propionyl}-erythromycin A (9E)-methoxymethylxime | 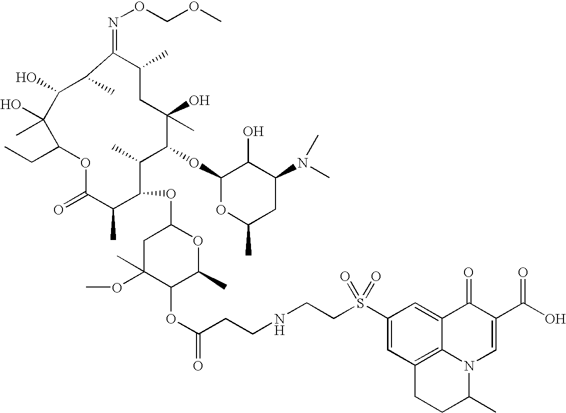 | 1198.0 |
| 10 | 2 + 7 | 4″-O-{[2-({2-[(2-carboxy-5-(*)-methyl-1-oxo-6,7-dihydro-1H,5H-pyrido[3,2,1-ij]quinolin-9-l)thio]ethyl}amino)ethyl]propionyl}-6-O-methylerythromycin A | 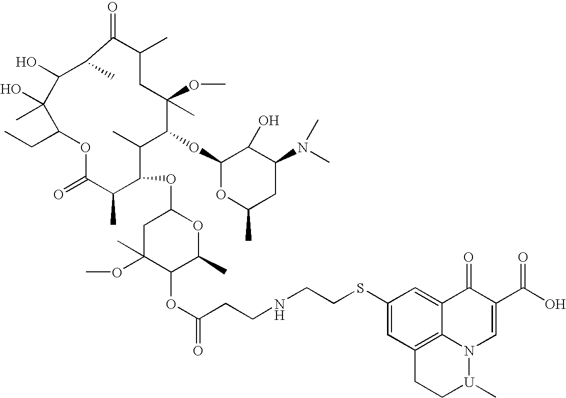 | 1121.1 |
| 11 | 3 + 7 | 4″-O-{[2-({2-[(2-carboxy-5-(*)-methyl-1-oxo-6,7-dihydro-1H,5H-pyrido[3,2,1-ij]quinolin-9-yl)thio]ethyl}amino)ethyl]propionyl}-6-O-methylerythromycin A | 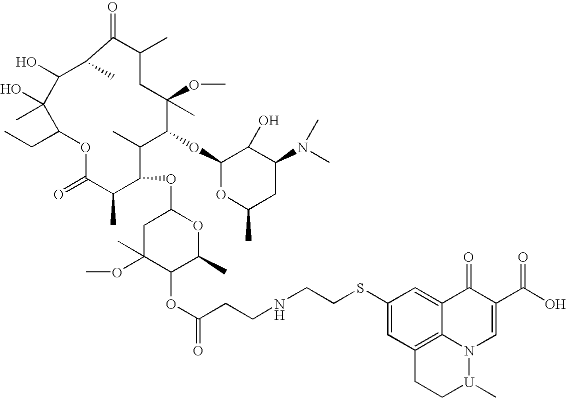 | 1121.1 |

### Example 12

### 4″-O-{[2-(2-{2-[(2-carboxy-5-(R,S)-methyl-1-oxo-6,7-dihydro-1H,5H-pyrido[3,2,1-ij]quinolin-9-yl)amino]ethyl}oxy(ethylamino))]propionyl}-6-O-methylerythromycin A

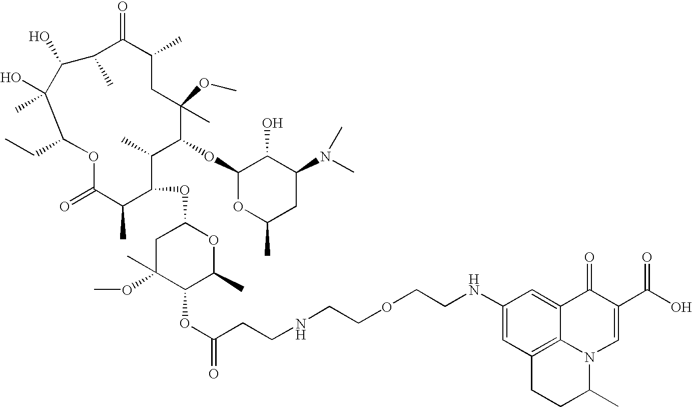

A solution of Intermediate 11 (0.07 g 0.2 mmol) and Intermediate 7 (0.14 g, 0.174 mmol) in DMSO (1 mL), triethylamine (0.15 mL) was heated at 80° C. for 22 h. The mixture was concentrated and purified by chromatography over silica gel eluting with 0-15% (9:1 MeOH/20M ammonia) in dichloromethane to give the title compound as a pale yellow solid (0.06 g); ESMS m/z 1149.2 [M+H]⁺.

### Example 13

### 4″-O-{[2-({2-[(2-carboxy-5-(R,S)-methyl-1-oxo-6,7-dihydro-1H,5H-pyrido[3,2,1-ij]quinolin-9-yl)amino]ethyl}amino)]propionyl}-6-O-methylerythromycin A

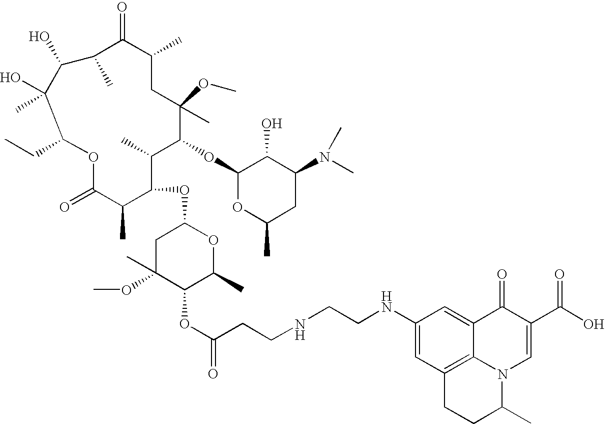

A solution of Intermediate 12 (0.13 g 0.37 mmol) and Intermediate 7 (0.23 g, 0.288 mmol) in DMSO (1.8 mL), triethylamine (0.28 mL) was heated at 80° C. for 16 h. The mixture was concentrated and purified by preparative reverse phase HPLC (MeCN/H₂O/0.1% HCO₂H eluent) followed by chromatography over silica gel eluting with 0-15% (9:1 MeOH/20M ammonia) in dichloromethane to give the title compound as a pale yellow solid (0.086 g). ESMS m/z 1104.1 [M+H]⁺.

### Example 14

### 4″-O-{[2-(2-{2-[(2-carboxy-5-(RS)-methyl-1-oxo-6,7-dihydro-1H,5H-pyrido[3,2,1-ij]quinolin-9-yl)amino]ethyl}oxy(ethylamino))]carbamoyl}-6-O-methylerythromycin A

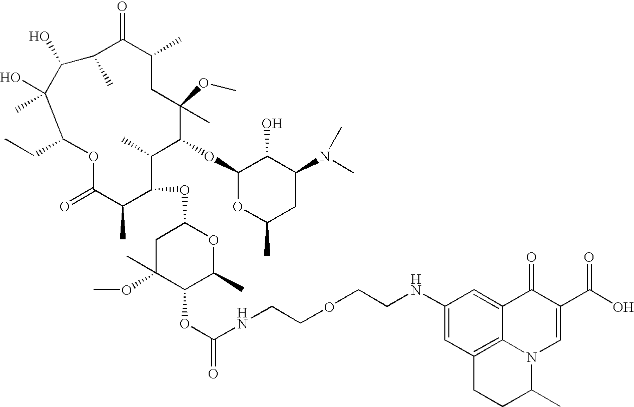

To a mixture of Intermediate 11 (0.165 g, 0.478 mmol) and Intermediate 13 (0.483 g, 0.572 mmol) in DMF (8 mL) was added DBU (0.8 mL). After stirring at room temperature for 65 h the mixture was concentrated and purified by preparative reverse phase HPLC (MeCN/H₂O/0.1% HCO₂H eluent) followed by chromatography over silica gel eluting with dichloromethane containing an increasing concentration of methanol/20M ammonia (0-10%) to yield the title compound as a yellow solid (0.235 g); ESMS m/z 1120.1 (MH⁺).

### Example 15

### 4″-O-{[2-({2-[(2-carboxy-5-(R,S)-methyl-1-oxo-6,7-dihydro-1H,5H-pyrido[3,2,1-ij]quinolin-9-yl)amino]ethyl}amino)]propionyl}-6-O-methylerythromycin A (9E)-methoxymethyloxime

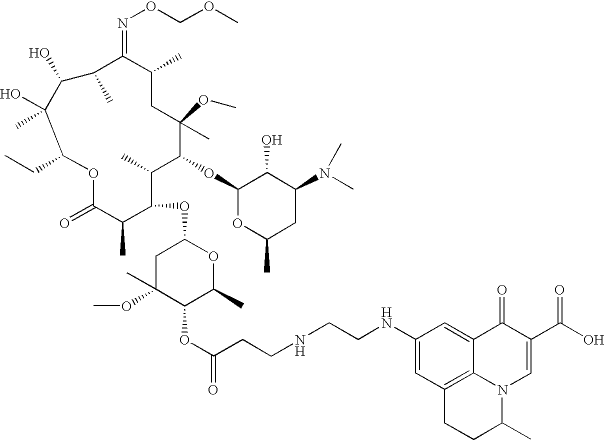

A solution of Intermediate 12 (0.151 g 0.43 mmol) and Intermediate 10 (0.283 g, 0.334 mmol) in DMSO (2 mL), triethylamine (0.325 mL) was heated at 80° C. for 16 h. The mixture was concentrated and purified by preparative reverse phase HPLC (MeCN/H₂O/0.1% HCO₂H eluent) followed by chromatography over silica gel eluting with 0-15% (9:1 MeOH/20M ammonia) in dichloromethane to give the title compound as a yellow solid (0.116 g). ESMS m/z 1149.1 [M+H]⁺.

### Example 16

### 4″-O-{[2-({2-[(2-carboxy-5-(R,S)-methyl-1-oxo-6,7-dihydro-1H,5H-pyrido[3,2,1-ij]quinolin-9-yl)amino]ethyl}amino)]propionyl}-6-O-methyl erythromycin A (9E)-oxime

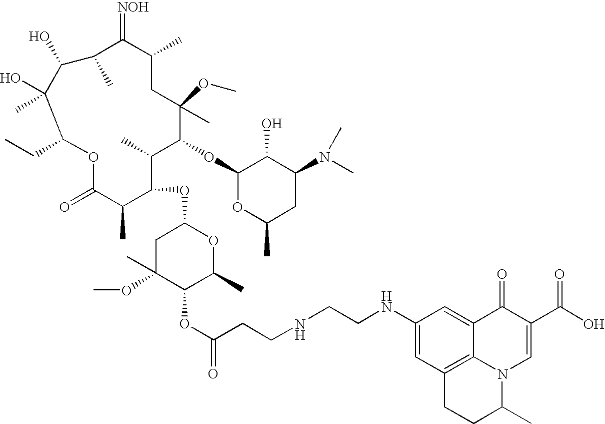

A solution of Intermediate 12 (0.151 g 0.43 mmol) and Intermediate 5 (0.267 g, 0.332 mmol) in DMSO (2 mL), triethylamine (0.325 mL) was heated at 80° C. for 16 h. The mixture was concentrated and purified by preparative reverse phase HPLC (MeCN/H₂O/0.1% HCO₂H eluent) followed by chromatography over silica gel eluting with 0-15% (9:1 MeOH/20M ammonia) in dichloromethane to give the title compound as a yellow solid (0.664 g). ESMS m/z 553.0 [M+2H]²⁺.

### Example 17

### 4″-O-{[(2-({2-[(2-carboxy-5-(R,S)-methyl-1-oxo-6,7-dihydro-1H,5H-pyrido[3,2,1-ij]quinolin-9-yl)thio]ethyl}amino)]ethyl}-6-O-methylerythromycin A

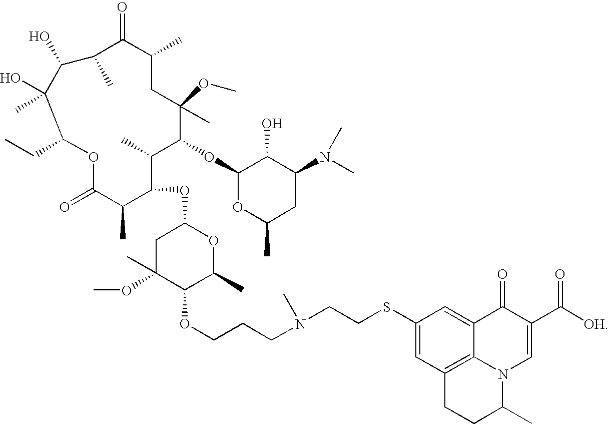

4″-O-Allyl-6-O-methyl-erythromycin A (95.8 g, 121 mmol) in dichloromethane (1 L) and methanol was cooled to −78° C. and TFA (18 mL) added. Ozonized oxygen was bubbled through until a blue colour developed (1.25 h). Argon was bubbled through the mixture to flush out the ozone, then dimethyl sulfide (35 mL) and triethylamine (50.4 mL) were added. The reaction was stirred at −78° C. for 30 min then removed from the cooling bath. After 0.5 h the reaction was warmed to 0° C. in a water bath and stirred for a further 0.5 h. The reaction mixture was washed with water (500 mL), dried and evaporated to dryness. The residue was dissolved in toluene and evaporated three times to give crude 4″-O-(2-oxoethyl)-6-O-methyl-erythromycin A, 103.7 g, which was used without purification.

This material (0.474 g, 0.6 mmol) in methanol (12 mL) was added to a suspension of Intermediate 1 (0.284 g, 0.8 mmol) and sodium acetate (0.148 mg, 1.8 mmol). To this resulting mixture was added acetic acid (0.09 mL), sodium cyanoborohydride (0.075 mg) and the mixture stirred at room temperature for 16 h. The reaction mixture was preabsorbed over silica gel then chromatographed over silica gel eluting with 0-15% (9:1 MeOH/20M ammonia) in dichloromethane to give the title compound as a white solid (0.19 g). ESMS m/z 1092.8 [M+H]⁺.

### Example 18

### 4″-O-{[2-({2-[(2-carboxy-5-(R,S)-methyl-1-oxo-6,7-dihydro-1H,5H-pyrido[3,2,1-ij]quinolin-9-yl)thio]ethyl}methylamino)]ethyl}-6-O-methyl erythromycin A

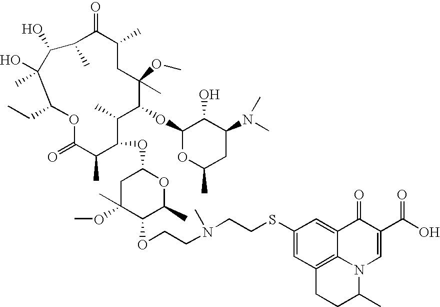

The title compound was also isolated from Example 17 after further purification by preparative reverse phase HPLC (MeCN/H₂O/0.1% HCO₂H eluent) followed by chromatography over silica gel eluting with 0-15% (9:1 MeOH/20M ammonia) in dichloromethane to give the title compound as a white solid (0.080g). ESMS m/z 1106.8 [M+H]⁺.

### Example 19

### 4″-O-{[2-({2-[(2-carboxy-5-(R,S)-methyl-1-oxo-6,7-dihydro-1H,5H-pyrido[3,2,1-ij]quinolin-9-yl)thio]ethyl}methylamino)]ethyl}-azithromycin

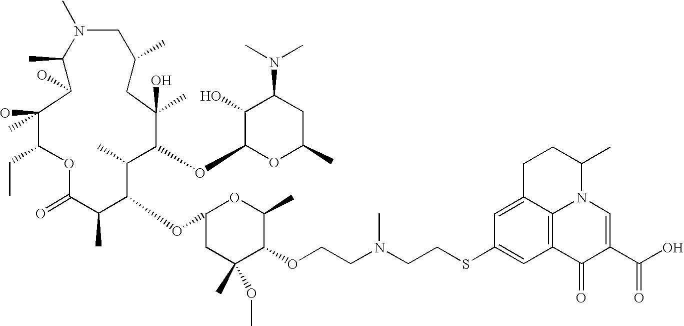

### a) 4″-O{[2-({2-[(2-carboxy-5-(R,S)-methyl-1-oxo-6,7-dihydro-1H,5H-pyrido[3,2,1-ij]quinolin-9-yl)thio]ethyl}amino)]ethyl}-azithromycin-11,12-carbonate

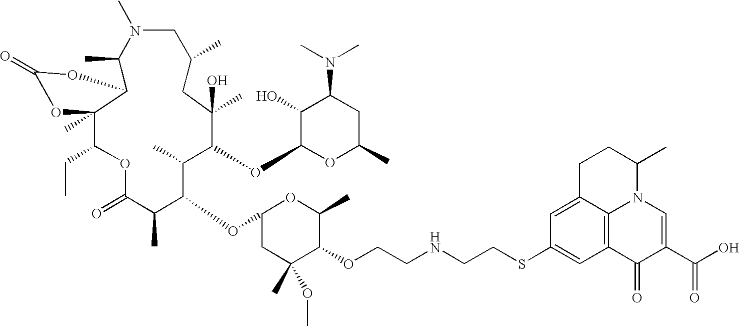

4″-O-Allyl-2′-O-acetyl-azithromycin-11,12-carbonate (9.0 g, 10.5 mmol) in dichloromethane (200 mL) and methanol (20 mL) was cooled to −78 C and trifluoroacetic acid (3.2 mL) added. Ozonized oxygen was bubbled through until a blue colour developed (1 h). Argon was bubbled through the mixture to flush out the ozone, then dimethyl sulfide (3.1 mL) and triethylamine (6.6 mL) were added. The reaction was stirred at −78° C. for 15 min then removed from the cooling bath and warmed to room temperature. The reaction mixture was washed successively with a saturated aqueous solution of sodium bicarbonate, water and brine then dried and evaporated to dryness to give crude 2′-O acetyl-4″-O-(2-oxoethyl)-azithromycin 11,12-carbonate, (9.5 g) which was used without purification.

This material (0.37 g, 0.43 mmol) in methanol (10 mL) was added to a suspension of Intermediate 1 (0.22 g, 0.69 mmol) and sodium acetate (0.113 mg, 1.37 mmol) in methanol (4 mL). To this resulting mixture was added acetic acid (0.068 mL), sodium cyanoborohydride (0.062 mg) and the mixture stirred at room temperature for 16 h. The reaction mixture was concentrated then purified by preparative reverse phase HPLC (MeCN/H₂O/0.1% HCO₂H eluent) to give the title compound as a white solid (0.145 g). ESMS m/z 560.6 [M+2H]²⁺.

### b) 4″-O-{[2-({2-[(2-carboxy-5-(R,S)-methyl-1-oxo-6,7-dihydro-1H,5H-pyrido[3,2,1-ij]quinolin-9-yl)thio]ethyl}methylamino)]ethyl}-azithromycin-11,12-carbonate

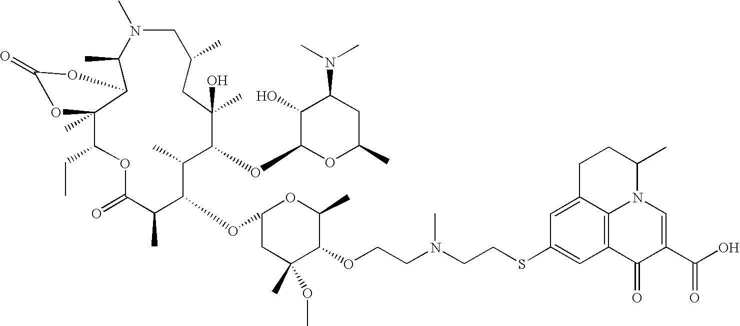

Example 19a (0.13 g, 0.12 mmol) in chloroform (1 mL) was treated with a 37% aqueous solution of formaldehyde (0.018 mL) and formic acid (0.015 mL). The suspension was heated to 60° C. for 2.5 h. The reaction mixture was chromatographed over silica gel eluting with 0-15% (9:1 MeOH/20M ammonia) in dichloromethane to give the title compound as a white solid (0.055 g). ESMS m/z 567.6 [M+2H]²⁺.

### c) 4″-O-{[2-({2-[(2-carboxy-5-(R,S)-methyl-1-oxo-6,7-dihydro-1H,5H-pyrido[3,2,1-ij]quinolin-9-yl)thio]ethyl}methylamino)]ethyl}-azithromycin

Example 19b (0.055 g, 0.043 mmol) in acetonitrile (0.7 mL) was treated with a 10% aqueous solution of potassium carbonate (2 mL) at 60° C. for 6 h then at 70° C. for 16 h. The acetonitrile was removed under vacuum and the residue partitioned between brine and dichloromethane. The organic layer was dried and evaporated to give the title compound as a white solid (0.042 g); ESMS m/z 554.7 [M+2H]²⁺.

### Example 20

### 4″-O-{[2-({2-[(2-Carboxy-5-(R,S)-methyl-1-oxo-6,7-dihydro-1H,5H-pyrido[3,2,1-ij]quinolin-9-yl)thio]ethyl}ethylamino)]ethyl}-azithromycin

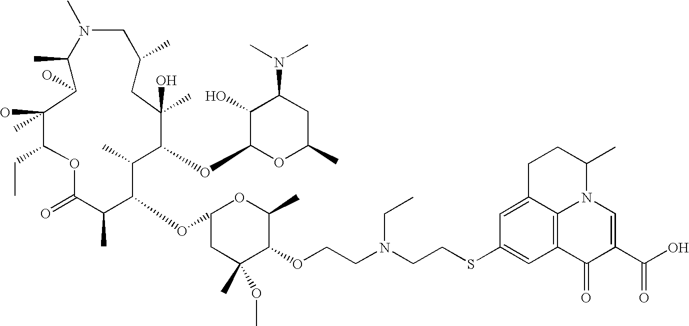

### a) 2′-O-Acetyl-4″-O-{[2-({2-[(2-carboxy-5-(R,S)-methyl-1-oxo-6,7-dihydro-1H,5H-pyrido[3,2,1-ij]quinolin-9-yl)thio]ethyl}amino)]ethyl}-azithromycin-11,12-carbonate Ethyl Ester

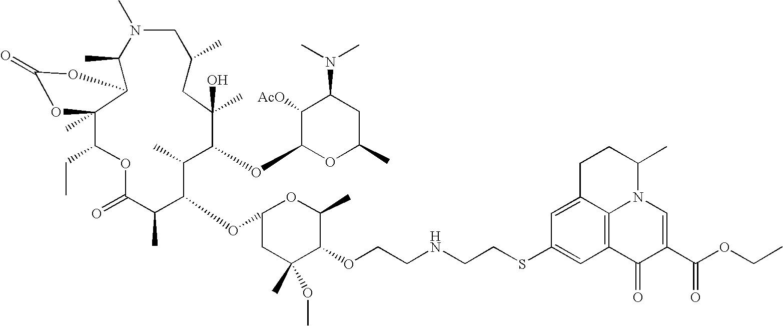

4″-O-Allyl-2′-O-acetyl-azithromycin-11,12-carbonate (9.0 g, 10.5 mmol) in dichloromethane (200 mL) and methanol (20 mL) was cooled to −78 C and trifluoroacetic acid (3.2 mL) added. Ozonized oxygen was bubbled through until a blue colour developed (1 h). Argon was bubbled through the mixture to flush out the ozone, then dimethyl sulfide (3.1 mL) and triethylamine (6.6 mL) were added. The reaction was stirred at −78° C. for 15 min then removed from the cooling bath and warmed to room temperature. The reaction mixture was washed successively with a saturated aqueous solution of sodium bicarbonate, water and brine then dried and evaporated to dryness to give crude 2′-O-acetyl-4″-O-(2-oxoethyl)-azithromycin 11,12-carbonate, (9.5 g) which was used without purification.

This material (0.644 g, 0.75 mmol) in dichloromethane (10 mL) was added to Intermediate 14b (0.374 g, 1 mmol) under argon. 3 A Molecular sieves (0.3 g) were added and after 5 min. sodium triacetoxy borohydride (0.32 g) was added, followed 5 min later by acetic acid (2 drops). After stirring at room temperature for 16 h the mixture was filtered, evaporated and the residue chromatographed eluting with 0-5% (10:1 methanol/20M ammonia) in dichloromethane to give the title compound (0.75 g); ESMS m/z 1218.0 [M+H]⁺.

### b) 4″-O-{[2-({2-[(2-Carboxy-5-(R,S)-methyl-1-oxo-6,7-dihydro-1H,5H-pyrido[3,2,1-ij]quinolin-9-yl)thio]ethyl}ethylamino)]ethyl}-azithromycin

Example 20a (0.75 g, 0.6 mmol) in acetonitrile (21 mL) was treated with a 10% aqueous solution of potassium carbonate (16 mL) at 70° C. for 16 h. The acetonitrile was removed under vacuum and the residue partitioned between brine and dichloromethane. The organic layer was dried and evaporated. The residue was dissolved in THF (6 mL) and treated with 0.5M aqueous solution of lithium hydroxide for 16 h. The reaction mixture was diluted with water and the pH adjusted to 5-6 by the addition of a 10% aqueous solution of citric acid. After the addition of dichloromethane the phases were separated and the organic layer was dried and evaporated. The residue was chromatographed eluting with 0-12% (10:1 methanol/20M ammonia) in dichloromethane to give the title compound as a pale yellow solid (0.18 g); ESMS m/z 561.7[M+2H]²⁺.

### Example 21

### 4″-O-{2-[2-(2-Carboxy-5-(R,S)-methyl-6,7-dihydro-1H,5H-pyrido[3,2,1-ij]-1-oxo-quinolin-9-ylthio)ethoxy]ethylcarbamoyl}-erythromycin A 9(E)-O-methoxymethyloxime

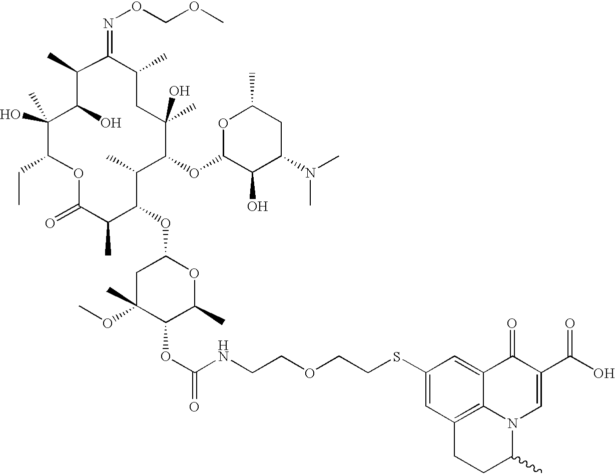

### a) 2′-O-Acetyl-4″-O-{2-[2-(2-carboxy-5-(R,S)-methyl-6,7-dihydro-1H,5H-pyrido[3,2,1-ij]-1-oxo-quinolin-9-ylthio)ethoxy]ethylcarbamoyl}-erythromycin A 9(E)-O-methoxymethyloxime

To a solution of Intermediate 19 (0.195 g) in dry DMF (3 mL) a solution of Intermediate 16 (0.100 g) and DBU (0.095 mL) in dry DMF (3 mL) was added dropwise at room temperature. The resulting mixture was stirred at 40° C. for 24 h. Solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid); the acetonitrile fraction was evaporated in vacuo and the resulting residue was purified by flash chromatography (silica gel, eluent 5-8% of 9/1 methanol/20M ammonia in dichloromethane) to give the title compound (0.125 g) as white foam; ESMS m/z 1223.3 [M+H]⁺.

### b) 4″-O-{2-[2-(2-Carboxy-5-(R,S)-methyl-6,7-dihydro-1H,5H-pyrido[3,2,1-ij]-1-oxo-quinolin-9-ylthio)ethoxy]ethylcarbamoyl}-erythromycin A 9(E)-O-methoxy Methyloxime

A solution of Example 21a (0.125 g) in MeOH (5 mL) was stirred at 50° C. overnight. The solvent was removed in vacuo to give the title compound (0.120 g); ESMS m/z 1181.9 [M+H]⁺.

### Example 22

### 4″-O-{2-[3-(2-Carboxy-5-(R,S)-methyl-1-oxo-6,7-dihydro-1H,5H-pyrido[3,2,1-ij]-quinolin-9-yl)propyloxy]ethylcarbamoyl}-6-O-methyl-erythromycin A

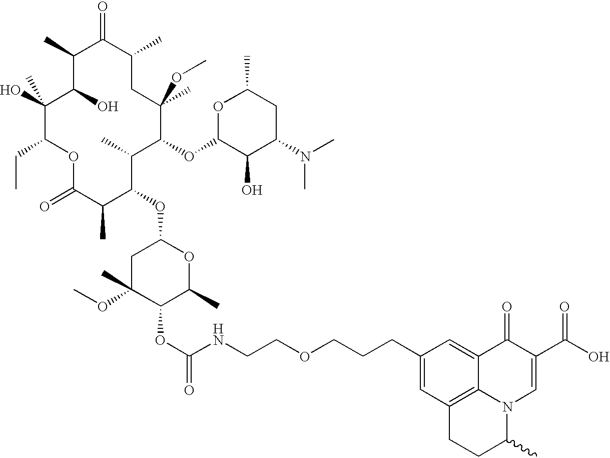

To a solution of Intermediate 13 (0.300g) a solution of Intermediate 15 (0.163 g) and DBU (0.160 mL) in DMF (5 mL) was added dropwise at room temperature. The resulting mixture was stirred at 50° C. for 24 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid); the acetonitrile fraction was evaporated in vacuo and the resulting residue was purified by flash chromatography (silica gel, eluent 0-10% of 9/1 methanol/20M ammonia in dichloromethane) to give the title compound (0.095 g) as white foam; ESMS m/z 1118.8 [M+H]⁺.

### Example 23

### 4″-O-{2-[3-(2-Carboxy-5-(R,S)-methyl-1-oxo-6,7-dihydro-1H,5H-pyrido[3,2,1-ij]-quinolin-9-yl)propyloxy]ethylcarbamoyl}-erythromycin A 9(E)-O-methoxymethyl Oxime

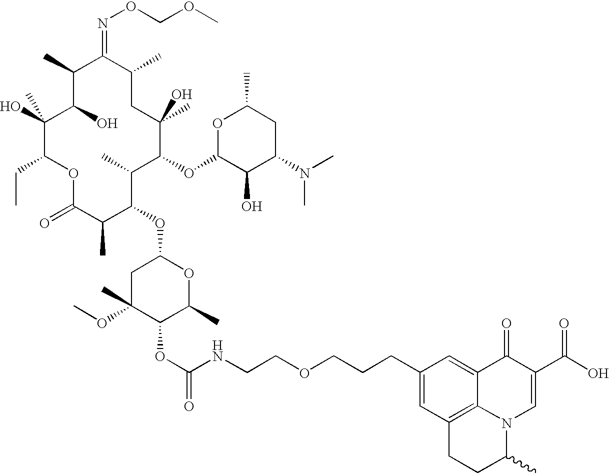

### a) 2′-O-Acetyl-4″-O-{2-[3-(2-carboxy-5-(R,S)-methyl-1-oxo-6,7-dihydro-1H,5H-pyrido[3,2,1-ij]-quinolin-9-yl)propyloxy]ethylcarbamoyl}-erythromycin A 9(E)-O-methoxymethyloxime

Intermediate 19 (0.300 g) was dissolved in dry DMF (4 mL) under nitrogen. To this solution a solution of Intermediate 15 (0.148 g) and DBU (0.145 mL) in dry DMF (4 mL) was added dropwise at room temperature. The resulting mixture was stirred at 40° C. for 24 h. Solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 0-5% of 9/1 methanol/20M ammonia in dichloromethane) to give the title compound (0.110 g) as white foam; ESMS m/z 1205.4 [M+H]⁺.

### b) 4″-O-{2-[3-(2-Carboxy-5-(R,S)-methyl-1-oxo-6,7-dihydro-1H,5H-pyrido[3,2,1-ij]-quinolin-9-yl)propyloxy]ethylcarbamoyl}-erythromycin A 9(E)-O-methoxymethyloxime

A solution of Example 24a (0.110 g) in MeOH (5 mL) was stirred at 50° C. overnight. The solvent was removed in vacuo to give the title compound (0.087 g); ESMS m/z 1164.0 [M+H]⁺.

### Example 24

### 4″-O-{2-[2-(2-Carboxy-5-(R,S)-methyl-6,7-dihydro-1H,5H-pyrido[3,2,1-ij]-1-oxo-quinolin-9-ylthio)ethoxy]ethylcarbamoyl}-6-O-methyl-erythromycin A

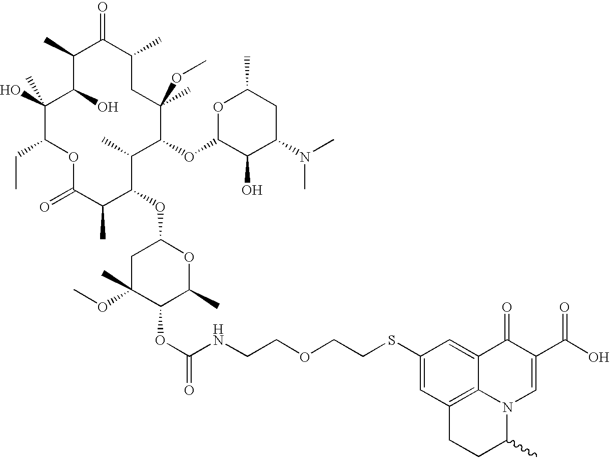

To a solution of Intermediate 13 (0.177 g) a solution of Intermediate 16 (0.100 g) and DBU (0.095 mL) in DMF (5 mL) was added dropwise at room temperature. The resulting mixture was stirred at 40° C. for 48 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 0-10% of 9/1 methanol/20M ammonia in dichloromethane); the resulting product was purified by preparative HPLC to obtain the title compound as trifluoroacetate salt. The residue was converted to the free base on silica gel cartridge (eluent eluent 10% of 9/1 methanol/20M ammonia in dichloromethane) to give the title compound (0.093 g); ESMS m/z 1136.7 [M+H]⁺.

### Example 25

### 4″-O-{2-[2-(2-Carboxy-5-(R,S)-methyl-6,7-dihydro-1H,5H-pyrido[3,2,1-ij]-1-oxo-quinolin-9-ylthio)ethoxy]ethylcarbamoyl}-erythromycin A 9(E)-oxime-

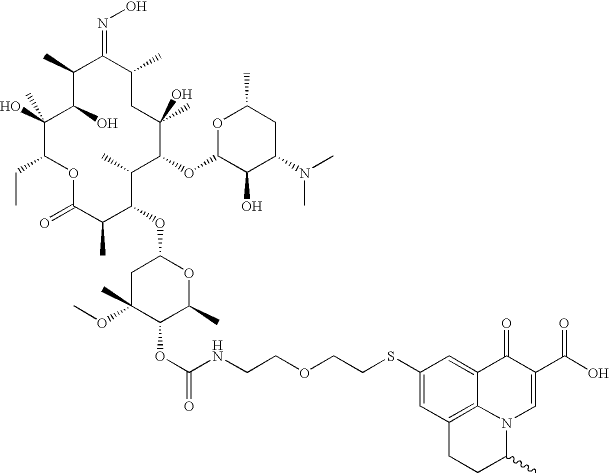

### a) 2′-O-Acetyl-4″-O-{2-[2-(2-carboxy-5-(R,S)-methyl-6,7-dihydro-1H,5H-pyrido[3,2,1-ij]-1-oxo-quinolin-9-ylthio)ethoxy]ethylcarbamoyl}-erythromycin A 9(E)-O-oxime

To a solution of Intermediate 20 (0.201 g) in dry DMF (4 mL) a solution of Intermediate 16 (0.100g) and DBU (0.095 mL) in dry DMF (4 mL) was added dropwise at room temperature. The resulting mixture was stirred at 40° C. for 20 h. Solvent was removed in vacuo and the residue was dissolved in MeOH and purified using a reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 5-12% of 9/1 methanol/20M ammonia in dichloromethane) to give the title compound (0.099 g) as white foam; ESMS m/z 1179.3 [M+H]⁺.

### b) 4″-O-{2-[2-(2-Carboxy-5-(R,S)-methyl-6,7-dihydro-1H,5H-pyrido[3,2,1-ij]-1-oxo-quinolin-9-ylthio)ethoxy]ethylcarbamoyl}-erythromycin A 9(E)-O-oxime

A solution of Example 26a (0.099 g) in MeOH (5 mL) was stirred at 50° C. overnight. The solvent was removed in vacuo to give the title compound (0.095 g); ESMS m/z 1138.0 [M+H]⁺.

### Example 26

### 4″-O-{2-[3-(2-Carboxy-5-(R,S)-methyl-1-oxo-6,7-dihydro-1H,5H-pyrido[3,2,1-ij]-quinolin-9-yl)propyloxy]ethylcarbamoyl}-azithromycin

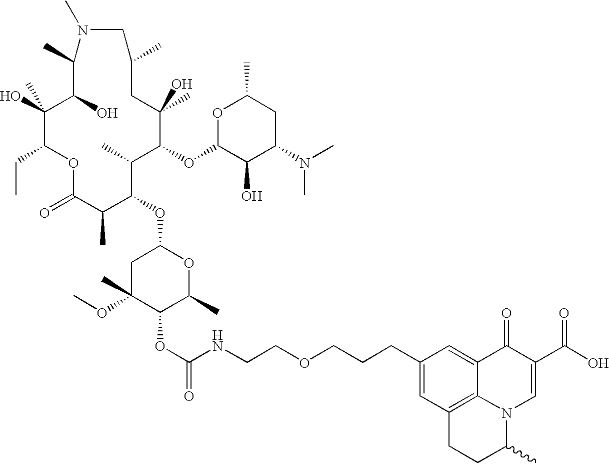

### a) 2′-O-Acetyl-4″-O-{2-[3-(2-carboxy-5-(R,S)-methyl-1-oxo-6,7-dihydro-1H,5H-pyrido[3,2,1-ij]-quinolin-9-yl)propyloxy]ethylcarbamoyl}-azithromycin

Intermediate 21 (0.300 g) was dissolved in dry DMF (4 mL) under nitrogen. To this solution a solution of Intermediate 15 (0.155 g) and DBU (0.152 mL) in dry DMF (4 mL) was added dropwise at room temperature. The resulting mixture was stirred at 40° C. for 24 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 0-10% of 9/1 methanol/20M ammonia in dichloromethane) to give the title compound (0.150 g) as white foam; ESMS m/z 1161.5 [M+H]⁺.

### b) 4″-O-{2-[3-(2-Carboxy-5-(R,S)-methyl-1-oxo-6,7-dihydro-1H,5H-pyrido[3,2,1-ij]-quinolin-9-yl)-propyloxy]-ethylcarbamoyl}-azithromycin

A solution of Example 26a (0.150 g) in MeOH (5 mL) was stirred at 50° C. overnight. The solvent was removed in vacuo to give the title compound (0.125 g); ESMS m/z 1119.9 [M+H]⁺.

### Example 27

### 4″-O-{2-[2-(2-Carboxy-5-(R,S)-methyl-6,7-dihydro-1H,5H-pyrido[3,2,1-ij]-1-oxo-quinolin-9-ylthio)ethoxy]ethylcarbamoyl}-azithromycin

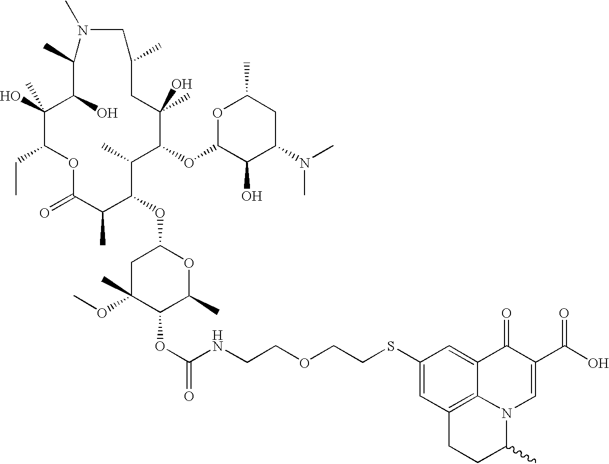

### a) 2′-O-Acetyl-4″-O-{2-[2-(2-carboxy-5-(R,S)-methyl-6,7-dihydro-1H,5H-pyrido[3,2,1-ij]-1-oxo-quinolin-9-ylthio)ethoxy]ethylcarbamoyl}-azithromycin

Intermediate 21 (0.300 g) was dissolved in dry DMF (4 mL) under nitrogen. To this solution a solution of Intermediate 16 (0.123 g) and DBU (0.150 mL) in dry DMF (4 mL) was added dropwise at room temperature. The resulting mixture was stirred at 40° C. for 24 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 0-10% of 9/1 methanol/20M ammonia in dichloromethane) to give the title compound (0.120 g) as white foam; ESMS m/z 1179.5 [M+H]⁺.

### b) 4″-O-{2-[2-(2-carboxy-5-(R,S)-methyl-6,7-dihydro-1H,5H-pyrido[3,2,1-ij]-1-oxo-quinolin-9-ylthio)ethoxy]ethylcarbamoyl}-azithromycin

A solution of Example 28a (0.120 g) in MeOH (5 mL) was stirred at 50° C. overnight. The solvent was removed in vacuo to give the title compound (0.105 g); ESMS m/z 1137.8 [M+H]⁺.

### Example 28

### 4″-O-{2-[2-(2-Carboxy-1-oxo-6,7-dihydro-1H,5H-pyrido[3,2,1-ij]-quinolin-9-yl)propyloxy]ethylcarbamoyl}-erythromycin A 9(E)-O-cyanomethyloxime

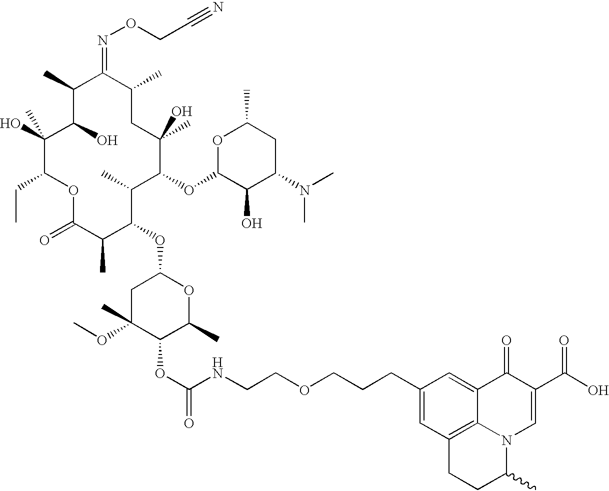

### a) 2′-O-Acetyl-4″-O-{2-[2-(2-Carboxy-5-(R,S)-methyl-1-oxo-6,7-dihydro-1H,5H-pyrido[3,2,1-ij]-quinolin-9-yl)propyloxy]ethylcarbamoyl}-erythromycin A 9(E)-O-cyanomethyloxime

To a solution of Intermediate 22 (0.300 g) in dry DMF (4 mL) a solution of Intermediate 15 (0.149 g) and DBU (0.145 mL) in dry DMF (4 mL) was added dropwise at room temperature. The resulting mixture was stirred at 40° C. for 24 h. The crude was purified using reverse phase chromatography (10 g C18 silica gel, reaction mixture charged as such on the cartridge, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 5-12% of 9/1 methanol/20M ammonia in dichloromethane) to give the title compound (0.130 g) as white foam; ESMS m/z 1200.6 [M+H]⁺.

### b) 4″-O-{2-[2-(2-Carboxy-5-(R,S)-methyl-1-oxo-6,7-dihydro-1H,5H-pyrido[3,2,1-ij]-quinolin-9-yl)propyloxy]ethylcarbamoyl}-erythromycin A 9(E)-O-cyanomethyl Oxime

A solution of Example 28a (0.130 g) in MeOH (5 mL) was stirred at 50° C. overnight. The solvent was removed in vacuo to give the title compound (0.115 g); ESMS m/z 1159.1 [M+H]⁺.

### Example 29

### 4″-O-{2-[3-(5-Carboxy-6-oxo-1,2-dihydro-6H-pyrrolo[3,2,1-ij]quinolin-8-yl)propyloxy]ethylcarbamoyl}-6-O-methyl Erythromycin A

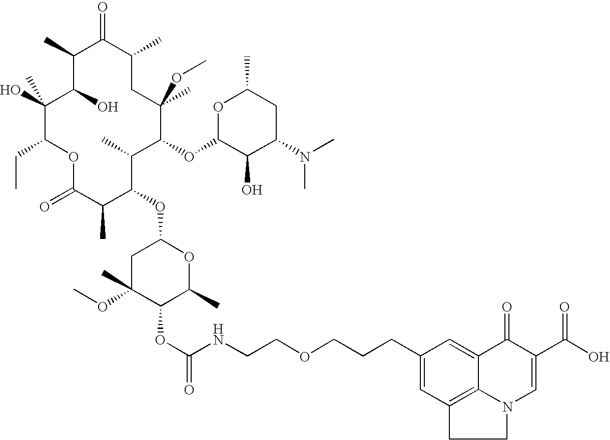

To a solution of Intermediate 13 (0.250 g) in DMF (5 mL) a solution of Intermediate 17 (0.128 g) and DBU (0.133 mL) in DMF (5 mL) was added dropwise at room temperature. The resulting mixture was stirred at 40° C. for 28 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 0-10% of 9/1 methanol/20M ammonia in dichloromethane) to give the title compound (0.180g); ESMS m/z 1090.9 [M+H]⁺.

### Example 30

### 4″-O-{2-[3-(5-Carboxy-6-oxo-1,2-dihydro-6H-pyrrolo[3,2,1-ij]quinolin-8-yl)propyloxy]ethylcarbamoyl}-erythromycin A 9(E)-O-methoxy Methyloxime

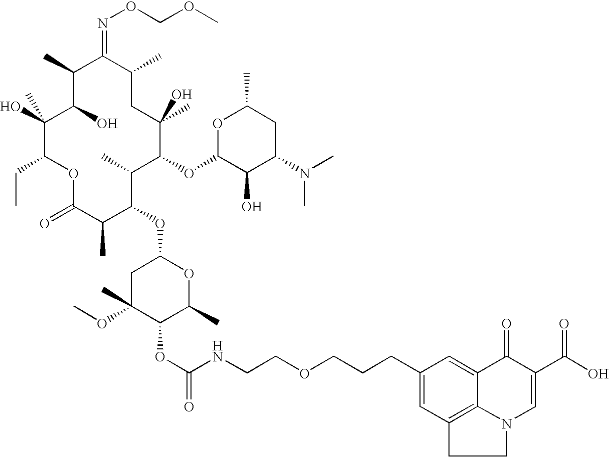

### a) 2′-O-Acetyl-4″-O-{2-[3-(5-carboxy-6-oxo-1,2-dihydro-6H-pyrrolo[3,2,1-ij]quinolin-8-yl)-propyloxy]-ethylcarbamoyl}-erythromycin A 9(E)-O-methoxy Methyloxime

Intermediate 19 (0.300 g) was dissolved in dry DMF (5 mL) under nitrogen. To this solution a solution of Intermediate 17 (0.139 g) and DBU (0.146 mL) in dry DMF (4 mL) was added dropwise at room temperature. The resulting mixture was stirred at 40° C. for 24 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 5-10% of 9/1 methanol/20M ammonia in dichloromethane) to give the title compound (0.285 g) as white foam; ESMS m/z 1177.3 [M+H]⁺.

### b) 4″-O-{2-[3-(5-carboxy-6-oxo-1,2-dihydro-6H-pyrrolo[3,2,1-ij]quinolin-8-yl)-propyloxy]-ethylcarbamoyl}-erythromycin A 9(E)-O-methoxy Methyloxime

A solution of Example 30a (0.285 g) in MeOH (5 mL) was stirred at 50° C. overnight. The solvent was removed in vacuo to give the title compound (0.265 g); ESMS m/z 1135.9 [M+H]⁺.

### Example 31

### 4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-methoxymethyloxime

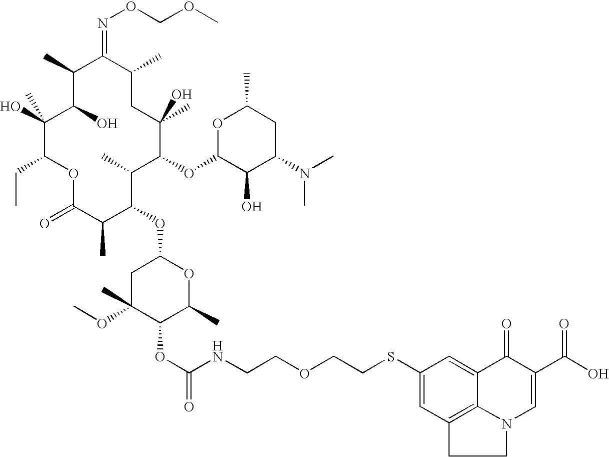

### a) 2′-O-Acetyl-4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-methoxymethyloxime

To a solution of Intermediate 19 (0.300 g) in dry DMF (4 mL) a solution of Intermediate 18 (0.097 g) and DBU (0.145 mL) in dry DMF (4 mL) was added dropwise at room temperature. The resulting mixture was stirred at 40° C. for 20 h. The solvent was removed in vacuo and the crude was dissolved in MeOH and purified using a reverse phase chromatography (10 g C18 silica gel, reaction mixture charged as such on the cartridge, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 0-10% of 9/1 methanol/20M ammonia in dichloromethane) to give the title compound (0.150g) as white foam; ESMS m/z 1195.3 [M+H]⁺.

### b) 4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-methoxy Methyloxime

A solution of Example 31a (0.150g) in MeOH (5 mL) was stirred at 50° C. overnight. The solvent was removed in vacuo to give the title compound (0.130g); ESMS m/z 1153.9 [M+H]⁺.

### Example 32

### 4″-O-{2-[2-(5-Carboxy-6-oxo-1,2-dihydro-6H-pyrrolo[3,2,1-ij]quinolin-8-ylthio)ethoxy]ethylcarbamoyl}-6-O-methyl-erythromycin A

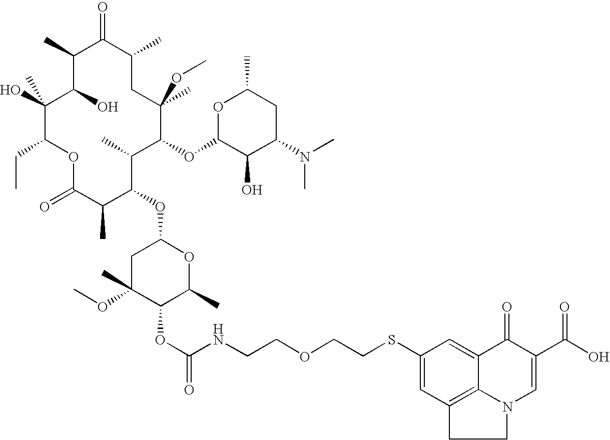

To a solution of Intermediate 13 (0.300 g) a solution of Intermediate 18 (0.119 g) and DBU (0.139 mL) in DMF (5 mL) was added dropwise at room temperature. The resulting mixture was stirred at 40° C. for 28 h. Solvent was removed in vacuo and the residue was dissolved in MeOH and purified using a reverse phase chromatography (10 g C18 silica gel, eluent 150 mL water/acetonitrile 95/5 with 0.5% formic acid; then 100 mL water/acetonitrile 5/95 with 0.5% formic acid); the acetonitrile fraction was evaporated in vacuo and the resulting residue was purified by flash chromatography (silica gel, eluent 0-10% of 9/1 methanol/20M ammonia in dichloromethane) to give the title compound (0.160 g); ESMS m/z 1108.8 [M+H]⁺.

### Example 33

### 4″-O-{2-[3-(5-Carboxy-6-oxo-1,2-dihydro-6H-pyrrolo-[3,2,1-ij]-quinolin-8-yl)propyloxy]ethylcarbamoyl}-azithromycin

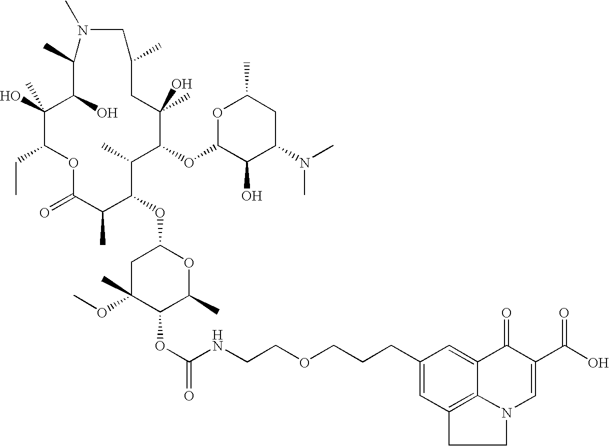

### a) 2′-O-Acetyl-4″-O-{2-[3-(5-Carboxy-6-oxo-1,2-dihydro-6H-pyrrolo-[3,2,1-ij]-quinolin-8-yl)propyloxy]ethylcarbamoyl}-azithromycin

Intermediate 21 (0.300 g) was dissolved in dry DMF (4 mL) under nitrogen. To this solution a solution of Intermediate 17 (0.146 g) and DBU (0.150 mL) in dry DMF (4 mL) was added dropwise at room temperature. The resulting mixture was stirred at 40° C. for 24 h. Solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid); the acetonitrile fraction was evaporated in vacuo and the resulting residue was purified by flash chromatography (silica gel, eluent 0-10% of 9/1 methanol/20M ammonia in dichloromethane) to give the title compound (0.175 g) as white foam; ESMS m/z 1133.4 [M+H]⁺.

### b) 4″-O-{2-[3-(5-Carboxy-6-oxo-1,2-dihydro-6H-pyrrolo-[3,2,1-ij]-quinolin-8-yl)propyloxy]ethylcarbamoyl}-azithromycin

A solution of Example 33a (0.175 g) in MeOH (5 mL) was stirred at 50° C. overnight. The solvent was removed in vacuo to give the title compound (0.170 g); ESMS m/z 1091.8 [M+H]⁺.

### Example 34

### 4″-O-{2-[2-(5-Carboxy-6-oxo-1,2-dihydro-6H-pyrrolo-[3,2,1-ij]-quinolin-8-ylthio)ethoxy]ethylcarbamoyl}-azithromycin

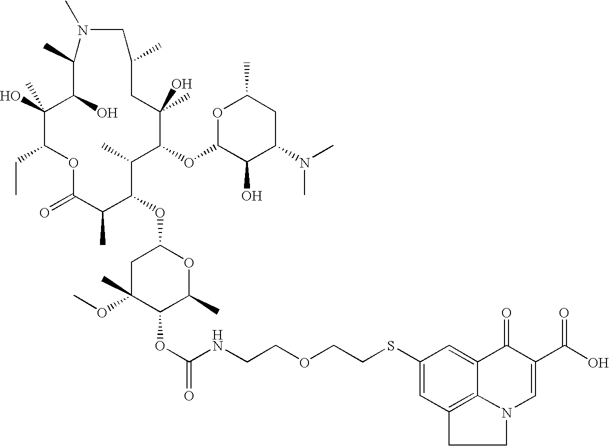

### a) 2′-O-Acetyl-4″-O-{2-[2-(5-Carboxy-6-oxo-1,2-dihydro-6H-pyrrolo-[3,2,1-ij]-quinolin-8-ylthio)ethoxy]ethylcarbamoyl}-azithromycin

Intermediate 21 (0.300 g) was dissolved in dry DMF (4 mL) under nitrogen. To this solution a solution of Intermediate 18 (0.114 g) and DBU (0.150 mL) in dry DMF (4 mL) was added dropwise at room temperature. The resulting mixture was stirred at 40° C. for 48 h. The solvent was removed in vacuo and the residue was dissolved in MeOH and purified using reverse phase chromatography (10 g C18 silica gel, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 0-10% of 9/1 methanol/20M ammonia in dichloromethane) to give the title compound (0.135 g) as white foam; ESMS m/z 1151.2 [M+H]⁺.

### b) 2′-O-Acetyl-4″-O-{2-[2-(5-Carboxy-6-oxo-1,2-dihydro-6H-pyrrolo-[3,2,1-ij]-quinolin-8-ylthio)ethoxy]ethylcarbamoyl}-azithromycin

A solution of Example 35a (0.135 g) in MeOH (5 mL) was stirred at 50° C. overnight. The solvent was removed in vacuo to give the title compound (0.130 g); ESMS m/z 1109.8 [M+H]⁺.

### Example 35

### 4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-oxime

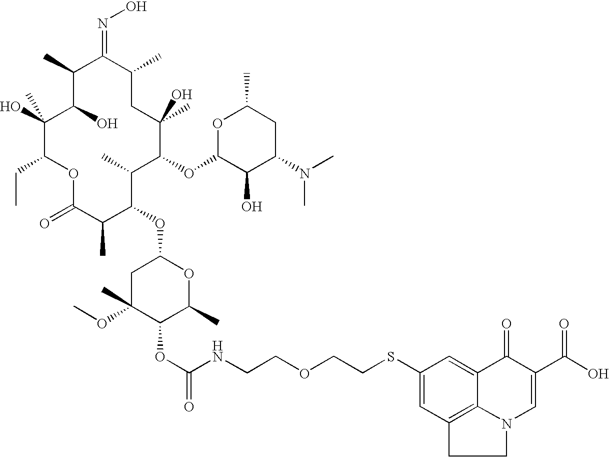

### a) 2′-O-Acetyl-4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-oxime

To a solution of Intermediate 20 (0.320 g) in dry DMF (4 mL) a solution of Intermediate 18 (0.101 g) and DBU (0.150 mL) in dry DMF (4 mL) was added dropwise at room temperature. The resulting mixture was stirred at 40° C. for 20 h. The solvent was removed and the crude was dissolved in MeOH and purified using reverse phase chromatography (10 g C18 silica gel, reaction mixture charged as such on the cartridge, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 0-10% of 9/1 methanol/20M ammonia in dichloromethane) to give the title compound (0.150 g) as white foam; ESMS m/z 1151.3 [M+H]⁺.

### b) 4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-oxime

A solution of Example 35a (0.150 g) in MeOH (5 mL) was stirred at 50° C. overnight. The solvent was removed in vacuo to give the title compound (0.140 g); ESMS m/z 1109.8 [M+H]⁺.

### Example 36

### 4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-cyanomethyloxime

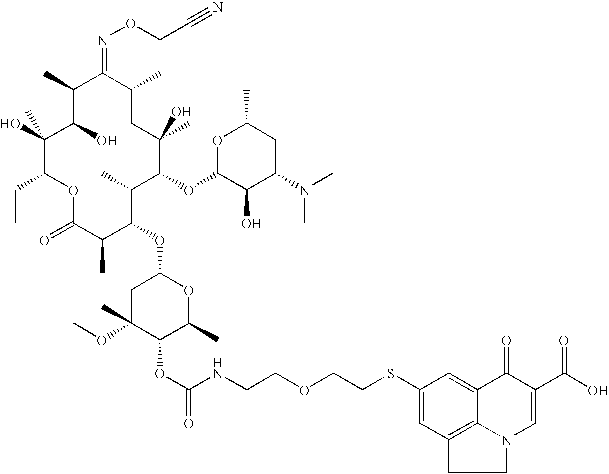

### a) 2′-O-Acetyl-4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-cyano Methyloxime

To a solution of Intermediate 22 (0.350 g) in dry DMF (4 mL) a solution of Intermediate 18 (0.126 g) and DBU (0.170 mL) in dry DMF (4 mL) was added dropwise at room temperature. The resulting mixture was stirred at 40° C. for 20 h. The crude was purified using reverse phase chromatography (10 g C18 silica gel, reaction mixture charged as such on the cartridge, eluent 150 mL water/acetonitrile 95/5 with 0.5% formic acid; then 100 mL water/acetonitrile 5/95 with 0.5% formic acid). The acetonitrile fraction was evaporated in vacuo and the resulting residue was purified by flash chromatography (silica gel, eluent 0-10% of 9/1 methanol/20M ammonia in dichloromethane) to give the title compound (0.167 g) as white foam; ESMS m/z 1190.5 [M+H]⁺.

### b) 2′-O-Acetyl-4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-cyano Methyloxime

A solution of Example 36a (0.167 g) in MeOH (5 mL) was stirred at 50° C. overnight. The solvent was removed in vacuo to give the title compound (0.152 g); ESMS m/z 1149.0 [M+H]⁺.

Biological Data

Using a standard broth dilution method in microtitre, compounds were tested for antibacterial activity. The compounds in the above examples gave minimum inhibitory concentrations (MICs) less than 1 microgram per millilitre against erythromycin-sensitive and erythromycin-resistant strains of Streptococcus pneumoniae and Streptococcus pyogenes.

However, it will appreciated by person skilled in the art that compounds of the invention may have different levels of activity against different strains of the same bacteria. Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A compound of formula (I) 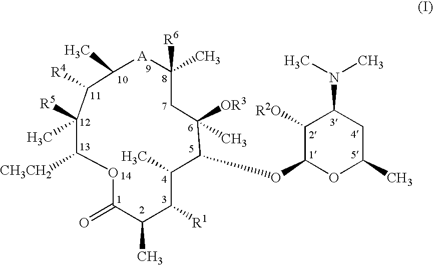
wherein
A is a bivalent radical —C(O)—, —N(R⁷)—CH₂—, —CH(NR⁸R⁹)— or —C(═NR¹⁰)—, or A and R⁴ taken together with the intervening atoms form a cyclic group having the following formula: 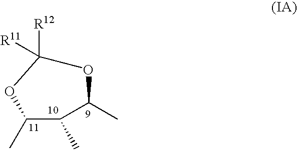
and R¹ is a group having the following formula: 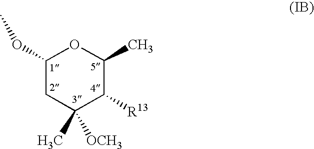
wherein R¹³ is —OC(O)(CH₂)_{d}U¹R¹⁴, —OC(O)N(R¹⁵)(CH₂)_{d}U¹R⁴, —O(CH₂)_{d}U¹R¹⁴, 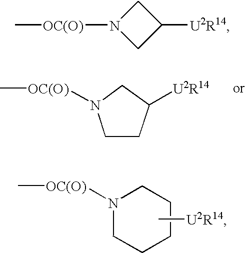
or
A is the bivalent radical —N(R⁷)—CH₂— and R¹ is a group having the following formula: 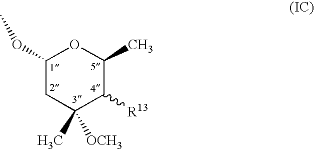
wherein R¹³ is —NHC(O)(CH₂)_{d}U¹R¹⁴;
R² is hydrogen or a hydroxyl protecting group;
R³ is hydrogen, C₁₋₄alkyl, or C₃₋₆alkenyl optionally substituted by 9- or 10-membered fused bicyclic heteroaryl;
R⁴ is hydroxy, C₃₋₆alkenyloxy optionally substituted by 9- or 10-membered fused bicyclic heteroaryl, or C₁₋₆alkoxy optionally substituted by C₁₋₆alkoxy or —O(CH₂)ₑNR⁷R¹⁶, or R⁴ and A taken together with the intervening atoms form a cyclic group of formula (IA),
R⁵ is hydroxy, or
R⁴ and R⁵ taken together with the intervening atoms form a cyclic group having the following formula: 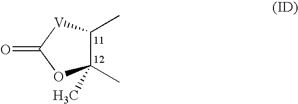
wherein V is a bivalent radical —CH₂—, —CH(CN)—, —O—, —N(R¹⁷)— or —CH(SR¹⁷)—, with the proviso that when R¹ is a group of formula (IC), V is —O—;
R⁶ is hydrogen or fluorine;
R⁷ is hydrogen or C₁₋₆alkyl;
R⁸ and R⁹ are each independently hydrogen, C₁₋₆alkyl or —C(O)R¹⁸, or
R⁸ and R⁹ together form ═CH(CR¹⁸R¹⁹)_{f}aryl, ═CH(CR¹⁸R¹⁹)_{f}heterocyclyl, ═CR¹⁸R¹⁹ or ═C(R¹⁸)C(O)OR¹⁸, wherein the alkyl, aryl and heterocyclyl groups are optionally substituted by up to three groups independently selected from R²⁰;
R¹⁰ is —OR²¹;
R¹¹ and R¹² are each independently hydrogen, C₁₋₆alkyl, heteroaryl, or aryl optionally substituted by one or two groups independently selected from hydroxyl and C₁₋₆alkoxy;
R¹⁴ is a heterocyclic group having the following formula: 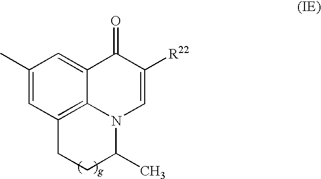
R¹⁵, R⁶, R¹⁸ and R¹⁹ are each independently hydrogen or C₁₋₆alkyl;
R¹⁷ is hydrogen or C₁₋₄alkyl optionally substituted by a group selected from optionally substituted phenyl, optionally substituted 5- or 6-membered heteroaryl and optionally substituted 9- or 10-membered fused bicyclic heteroaryl;
R²⁰ is halogen, cyano, nitro, trifluoromethyl, azido, —C(O)R²³, —C(O)OR²³, —OC(O)R²³, —OC(O)OR²³, —NR²⁴C(O)R²⁵, —C(O)NR²⁴R²⁵, —NR²⁴R²⁵, hydroxy, C₁₋₆alkyl, —S(O)ₕC₁₋₆-alkyl, C₁₋₆alkoxy, —(CH₂)ᵢaryl or —(CH₂)ᵢheteroaryl, wherein the alkoxy group is optionally substituted by up to three groups independently selected from —NR¹⁸R¹⁹, halogen and —OR¹⁸, and the aryl and heteroaryl groups are optionally substituted by up to five groups independently selected from halogen, cyano, nitro, trifluoromethyl, azido, —C(O)R¹⁶, —C(O)OR¹¹, —OC(O)OR²⁶, —NR²⁷C(O)R²⁸, —C(O)NR²⁷R²⁸, —NR²⁷R²⁸, hydroxy, C₁₋₆alkyl and C₁₋₆alkoxy;
R²¹ is hydrogen, C₁₋₆alkyl, C₃₋₇cycloalkyl, C₃₋₆alkenyl or a 5- or 6-membered heterocyclic group, wherein the alkyl, cycloalkyl, alkenyl and heterocyclic groups are optionally substituted by up to three groups independently selected from optionally substituted 5- or 6-membered heterocyclic group, optionally substituted 5- or 6-membered heteroaryl, —OR²⁹, —S(O)ⱼR²⁹, —NR²⁹R³⁰, —CONR²⁹R³⁰, halogen and cyano;
R²² is —C(O)OR³¹, —C(O)NHR³¹, —C(O)CH₂NO₂ or —C(O)CH₂SO₂R⁷;
R²³ is hydrogen, C₁₋₁₀alkyl, —(CH₂)ₖaryl or —(CH₂)ₖheteroaryl;
R²⁴ and R²⁵ are each independently hydrogen, —OR¹⁸, C₁₋₆alkyl, —(CH₂)ₘaryl or —(CH₂)ₘheterocyclyl;
R²⁶ is hydrogen, C₁₋₁₀alkyl, —(CH₂)ₙaryl or —(CH₂)ₙheteroaryl;
R²⁷ and R²⁸ are each independently hydrogen, —OR¹⁸, C₁₋₆alkyl, —(CH₂)ₚaryl or —(CH₂)ₚheterocyclyl;
R²⁹ and R³⁰ are each independently hydrogen, C₁₋₄alkyl or C₁₋₄alkoxyC₁₋₄alkyl;
R³¹ is hydrogen,
C₁₋₆alkyl optionally substituted by up to three groups independently selected from halogen, cyano, C₁₋₄alkoxy optionally substituted by phenyl or C₁₋₄alkoxy, —C(O)C₁₋₆alkyl, —C(O)OC₁₋₆alkyl, —OC(O)C₁₋₆alkyl, —OC(O)OC₁₋₆alkyl, —C(O)NR³²R³³, —NR³²R³³ and phenyl optionally substituted by nitro or —C(O)OC₁₋₆alkyl,
—(CH₂)_{q}C₃₋₇cycloalkyl,
—(CH₂)_{q}heterocyclyl,
—(CH₂)_{q}heteroaryl,
—(CH₂)_{q}aryl,
C₃₋₆alkenyl, or
C₃₋₆alkynyl;
R³² and R³³ are each independently hydrogen or C₁₋₆alkyl optionally substituted by phenyl or —C(O)OC₁₋₆alkyl, or
R³² and R³³, together with the nitrogen atom to which they are bound, form a 5- or 6-membered heterocyclic group optionally containing one additional heteroatom selected from oxygen, sulfur and N—R³⁴;
R³⁴ is hydrogen or methyl;
R³⁵ is hydrogen, C₁₋₄alkyl, C₃₋₇cycloalkyl, optionally substituted phenyl or benzyl, acetyl or benzoyl;
U¹ is a bivalent radical —W(CH₂)ᵣX—, —W(CH₂)ᵣ—, —W(CH₂)ᵣX(CH₂)ₛY—, —W(CH₂)ᵣX(CH₂)ₛ—, —W(CH₂)ᵣX(CH₂)ₛY(CH₂)ₜZ- or —W(CH₂)ᵣX(CH₂)ₛY(CH₂)ₜ—;
U² is U¹ or a bivalent radical —O—, —N(R³⁵)—, —S(O)ₙ— or —CH₂—;
W, X, Y and Z are each independently a bivalent radical —N(R³⁵)—, —O—, —S(O)ᵤ—, —N(R³⁵)C(O)—, —C(O)N(R³⁵)— or —N[C(O)R³⁵]—;
d is an integer from 2 to 5;
e is an integer from 2 to 4;
f, i, k, m, n, p and q are each independently integers from 0 to 4;
g is 0 or 1;
h, j and u are each independently integers from 0 to 2;
r, s and t are each independently integers from 2 to 5;
or a pharmaceutically acceptable derivative thereof.

2. A compound according to claim 1 wherein A is —C(O)—, —N(R⁷)—CH₂— or —C(═NR¹⁰)—.

3. A compound according to claim 1 wherein R¹ is

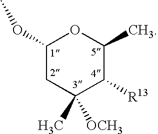

4. A compound according to claim 1 wherein U¹ is
—W(CH₂)ᵣX—.

5. A compound according to claim 1 wherein r is 2.

6. A compound according to claim 1 wherein R¹⁴ is a heterocyclic group having the following formula:

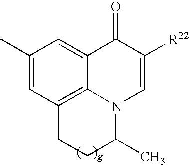

7. A compound according to claim 1 as defined in any one of Examples 1 to 11, or a pharmaceutically acceptable derivative thereof.

8. A compound selected from:
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-6-O-methyl-lerythromycin A,
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-azithromycin,
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-azithromycin-11,12-carbonate,
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-erythromycin A (9E)-oxime,
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)ethyl]propionyl}-erythromycin A (9E)-methoxymethyloxime,
4″-O-{[2-({2-[(2-carboxy-5-(R,S)-methyl-1-oxo-6,7-dihydro-1H,5H-pyrido[3,2,1-ij]quinolin-9-yl)sulfonyl]ethyl}amino)ethyl]propionyl}-6-O-methyl-erythromycin A,
4″-O-{[2-({2-[(2-carboxy-5-(R,S)-methyl-1-oxo-6,7-dihydro-1H,5H-pyrido[3,2,1-ij]quinolin-9-yl)sulfonyl]ethyl}amino)ethyl]propionyl}-azithromycin,
4″-O-{[2-({2-[(2-carboxy-5-(R,S)-methyl-1-oxo-6,7-dihydro-1H,5H-pyrido[3,2,1-ij]quinolin-9-yl)sulfonyl]ethyl}amino)ethyl]propionyl}-erythromycin A (9E)-methoxime,
4″-O-{[2-({2-[(2-carboxy-5-(R,S)-methyl-1-oxo-6,7-dihydro-1H,5H-pyrido[3,2,1-ij]quinolin-9-yl)sulfonyl]ethyl}amino)ethyl]propionyl}-erythromycin A (9E)-methoxymethyloxime,
4″-O-{[2-({2-[(2-carboxy-5-(*)-methyl-1-oxo-6,7-dihydro-1H,5H-pyrido[3,2,1-ij]quinolin-9-l)thio]ethyl}amino)ethyl]propionyl}-6-O-methyl-erythromycin A,
4″-O-{[2-({2-[(2-carboxy-5-(*)-methyl-1-oxo-6,7-dihydro-1H,5H-pyrido[3,2,1-ij]quinolin-9-yl)thio]ethyl}amino)ethyl]propionyl}-6-O-methyl-erythromycin A,
4″-O-{[2-(2-{2-[(2-carboxy-5-(R,S)-methyl-1-oxo-6,7-dihydro-1H,5H-pyrido[3,2,1-ij]quinolin-9-yl)amino]ethyl}oxy(ethylamino))]propionyl}-6-O-methyl-erythromycin A,
4″-O-{[2-({2-[(2-carboxy-5-(R,S)-methyl-1-oxo-6,7-dihydro-1H,5H-pyrido[3,2,1-ij]quinolin-9-yl)amino]ethyl}amino)]propionyl}-6-O-methyl-erythromycin A,
4″-O-{[2-(2-{2-[(2-carboxy-5-(R,S)-methyl-1-oxo-6,7-dihydro-1H,5H-pyrido[3,2,1-ij]quinolin-9-yl)amino]ethyl}oxy(ethylamino))]carbamoyl}-6-O-methyl-erythromycin A,
4″-O-{[2-({2-[(2-carboxy-5-(R,S)-methyl-1-oxo-6,7-dihydro-1H,5H-pyrido[3,2,1-ij]quinolin-9-yl)amino]ethyl}amino)]propionyl}-6-O-methyl-erythromycin A (9E)-methoxymethyloxime,
4″-O-{[2-({2-[(2-carboxy-5-(R,S)-methyl-1-oxo-6,7-dihydro-1H,5H-pyrido[3,2,1-ij]quinolin-9-yl)amino]ethyl}amino)]propionyl}-6-O-methyl-erythromycin A (9E)-oxime,
4″-O-{[2-({2-[(2-carboxy-5-(R,S)-methyl-1-oxo-6,7-dihydro-1H,5H-pyrido[3,2,1-ij]quinolin-9-yl)thio]ethyl}amino)]ethyl}-6-O-methyl-erythromycin A,
4″-O-{[2-({2-[(2-carboxy-5-(R,S)-methyl-1-oxo-6,7-dihydro-1H,5H-pyrido[3,2,1-ij]quinolin-9-yl)thio]ethyl}methylamino)]ethyl}-6-O-methyl-erythromycin A,
4″-O-{[2-({2-[(2-carboxy-5-(R,S)-methyl-1-oxo-6,7-dihydro-1H,5H-pyrido[3,2,1-ij]quinolin-9-yl)thio]ethyl}methylamino)]ethyl}-azithromycin,
4″-O-{[2-({2-[(2-Carboxy-5-(R,S)-methyl-1-oxo-6,7-dihydro-1H,5H-pyrido[3,2,1-ij]quinolin-9-yl)thio]ethyl}ethylamino)]ethyl}-azithromycin,
4″-O-{2-[2-(2-Carboxy-5-(R,S)-methyl-6,7-dihydro-1H,5H-pyrido[3,2,1-ij]-1-oxo-quinolin-9-ylthio)ethoxy]ethylcarbamoyl}-erythromycin A 9(E)-O-methoxymethyloxime,
4″-O-{2-[3-(2-Carboxy-5-(R,S)-methyl-1-oxo-6,7-dihydro-1H,5H-pyrido[3,2,1-ij]-quinolin-9-yl)propyloxy]ethylcarbamoyl}-6-O-methyl-erythromycin A,
4″-O-{2-[3-(2-Carboxy-5-(R,S)-methyl-1-oxo-6,7-dihydro-1H,5H-pyrido[3,2,1-ij]-quinolin-9-yl)propyloxy]ethylcarbamoyl}-erythromycin A 9(E)-O-methoxymethyl oxime,
4″-O-{2-[2-(2-Carboxy-5-(R,S)-methyl-6,7-dihydro-1H,5H-pyrido[3,2,1-ij]-1-oxo-quinolin-9-ylthio)ethoxy]ethylcarbamoyl}-6-O-methyl-erythromycin A,
4″-O-{2-[2-(2-Carboxy-5-(R,S)-methyl-6,7-dihydro-1H,5H-pyrido[3,2,1-ij]-1-oxo-quinolin-9-ylthio)ethoxy]ethylcarbamoyl}-erythromycin A 9(E)-oxime,
4″-O-{2-[3-(2-Carboxy-5-(R,S)-methyl-1-oxo-6,7-dihydro-1H,5H-pyrido[3,2,1-ij]-quinolin-9-yl)propyloxy]ethylcarbamoyl}-azithromycin,
4″-O-{2-[2-(2-Carboxy-5-(R,S)-methyl-6,7-dihydro-1H,5H-pyrido[3,2,1-ij]-1-oxo-quinolin-9-ylthio)ethoxy]ethylcarbamoyl}-azithromycin,
4″-O-{2-[2-(2-Carboxy-1-oxo-6,7-dihydro-1H,5H-pyrido[3,2,1-ij]-quinolin-9-yl)propyloxy]ethylcarbamoyl}-erythromycin A 9(E)-O-cyanomethyl oxime,
4″-O-{2-[3-(5-Carboxy-6-oxo-1,2-dihydro-6H-pyrrolo[3,2,1-ij]quinolin-8-yl)propyloxy]ethylcarbamoyl}-6-O-methyl-erythromycin A,
4″-O-{2-[3-(5-Carboxy-6-oxo-1,2-dihydro-6H-pyrrolo[3,2,1-ij]quinolin-8-yl)propyloxy]ethylcarbamoyl}-erythromycin A 9(E)-O-methoxy methyloxime,
4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-methoxy methyloxime,
4″-O-{2-[2-(5-Carboxy-6-oxo-1,2-dihydro-6H-pyrrolo[3,2,1-ij]quinolin-8-ylthio)ethoxy]ethylcarbamoyl}-6-O-methyl-erythromycin A,
4″-O-{2-[3-(5-Carboxy-6-oxo-1,2-dihydro-6H-pyrrolo-[3,2,1-ij]-quinolin-8-yl)propyloxy]ethylcarbamoyl}-azithromycin,
4″-O-{2-[2-(5-Carboxy-6-oxo-1,2-dihydro-6H-pyrrolo-[3,2,1-ij]-quinolin-8-ylthio)ethoxy]ethylcarbamoyl}-azithromycin,
4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-oxime, and
4″-O-{2-[2-(5-Carboxy-6-oxo-1,2-dihydro-6H-pyrrolo[3,2,1-ij]quinolin-8-ylthio)ethoxy]ethylcarbamoyl}-erythromycin A 9(E)-O-cyano methyloxime,
and pharmaceutically acceptable derivatives thereof.

9. A process for the preparation of a compound as claimed in claim 1, or a pharmaceutically acceptable derivative thereof, which comprises reacting a compound of formula (XII), wherein R² is optionally a hydroxyl protecting group, 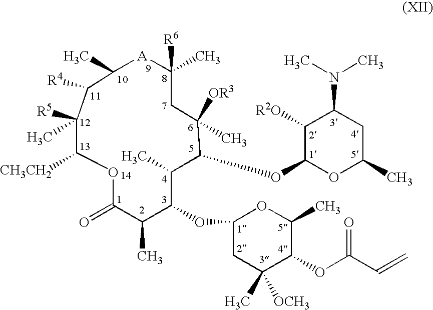
with a compound of formula HU^{1z}R^{14z} (VIII) wherein R^{14z} is R¹⁴ as defined in claim 1 or a group convertible to R¹⁴ and U^{1z} is —W(CH₂)ᵣX or —W(CH₂)ᵣ— or a group convertible to —W(CH₂)ᵣX— or —W(CH₂)ᵣ— in which W is —N(R³⁵)— or —S—, to produce a compound of formula (I) wherein d is 2 and W is —N(R³⁵)— or —S—, and thereafter, if required, subjecting the resulting compound to one or more of the following operations:
i) removal of the protecting group R²,
ii) conversion of U^{1z}R^{14z} to U¹R¹⁴, and
iii) conversion of the resultant compound of formula (I) into a pharmaceutically acceptable derivative thereof.

10. A compound as claimed in claim 1, or a pharmaceutically acceptable derivative thereof, for use in therapy.

11. A compound as claimed in claim 1, or a pharmaceutically acceptable derivative thereof, for use in the treatment or prophylaxis of systemic or topical microbial infections in a human or animal body.

12. (canceled)

13. A method for the treatment of the human or non-human animal body to combat microbial infection comprising administration to a body in need of such treatment of an effective amount of a compound as claimed in claim 1, or a pharmaceutically acceptable derivative thereof.

14. A pharmaceutical composition comprising a compound as claimed in claim 1, or a pharmaceutically acceptable derivative thereof, in association with a pharmaceutically acceptable excipient, diluent and/or carrier.
